(19) United States
(12) Patent Application Publication
(10) Pub. No.: US 2019/0355454 A1
(43) Pub. Date: Nov. 21, 2019

(54) GOAL BASED THERAPY OPTIMIZATION FOR PATIENT

(71) Applicant: Opya, Inc., San Mateo, CA (US)

(72) Inventors: Suchitra Deshpande, San Carlos, CA (US); Jessica Hammond Owens, San Francisco, CA (US); Afton Kerry Vechery, San Francisco, CA (US); Jonathan Kenneth Wright, San Francisco, CA (US)

(21) Appl. No.: 16/528,433

(22) Filed: Jul. 31, 2019

Related U.S. Application Data
(63) Continuation-in-part of application No. 16/409,721, filed on May 10, 2019.
(60) Provisional application No. 62/669,725, filed on May 10, 2018.
(60) Provisional application No. 62/669,748, filed on May 10, 2018.

(51) Int. Cl. G16H 10/60; G16H 50/20

(52) U.S. Cl. CPC G16H 10/60; G16H 50/20

(57) ABSTRACT

A management platform for characterizing one or more neuropsychiatric and/or neurological disorders of a patient based on received assessment data and heuristic data, which is employed to generate a diagnosis, goals and therapies for the patient. Machine learning models are used to optimize the diagnosis, personalized goals, and therapies provided for the patient. Different types of assessment data may include: layperson data, clinical data, biometric data, video data, medical data, heuristic data, or the like. Also, different types of the assessment data may be weighted differently.

### CROSS-REFERENCE TO RELATED APPLICATIONS

This Utility Patent Application is a Continuation-in-Part of U.S. patent application Ser. No. 16/409,721 filed on May 10, 2019, which is based on previously filed U.S. Provisional Patent Application Ser. No. 62/669,725 filed on May 10, 2018, and U.S. Provisional Patent Application Ser. No. 62/669,748 filed on May 10, 2018, the benefit of the filing date of which is hereby claimed under 35 U.S.C. § 119(e) and § 120 and the contents of which are each further incorporated in entirety by reference.

### TECHNICAL FIELD

This invention relates generally to the neurology field, and more specifically to a new and useful method for characterizing disorders.

### BACKGROUND

Neuropsychiatry is a specialty of medicine crossing neurology and psychology, which entails mental disorders attributable to diseases of the nervous system. While many neuropsychiatric and neurological disorders are treatable, success of a treatment regimen relies heavily upon early diagnosis, identification of symptoms during key periods of development, accurate diagnosis, and formation of personalized goals and therapies that match the patient's diagnosis for one or more disorders. These neuropsychiatric and/or neurological disorders may include Autism Spectrum Disorder, mental retardation, or the like.

Unfortunately, current standards of diagnosis and treatment are responsible for unrealistic goals for improving disorders, delays in diagnoses of disorders and/or misdiagnoses of disorders, which cause the disorders to remain untreated or undertreated. While the delays are partially due to the non-intuitive, time-sensitive, and patient demographic-sensitive nature of such disorders, current standards of diagnosis are unnecessarily deficient in many aspects. Also, the current standards of diagnosis can be difficult to administer due to inherent differences between a diagnosis environment and a patient's natural environment. Additionally, these inherent deficiencies, further limitations in diagnosis, treatment, and/or monitoring of patient progress during treatment prevent adequate care of patients with diagnosable and treatable disorders.

Machine learning is increasingly playing a larger and more important role in developing and improving the understanding of complex patient disorders. As machine learning techniques have matured, machine learning has rapidly moved from the theoretical to the practical. Combined with the advent of big-data technology, machine learning solutions are being applied to a variety of industries and applications that until now were difficult, if not impossible to effectively reason about. As such, there has been a need for the development of different types of machine learning models that may be used for diagnosis, identifying personalized goals and therapies, and predicting treatment outcomes for the therapies for different patient disorders.

### BRIEF DESCRIPTION OF THE DRAWINGS

Non-limiting and non-exhaustive embodiments of the present innovations are described with reference to the following drawings. In the drawings, like reference numerals refer to like parts throughout the various figures unless otherwise specified. For a better understanding of the described innovations, reference will be made to the following Detailed Description of the Various Embodiments, which is to be read in association with the accompanying drawings, wherein:

FIG. 1 illustrates a system environment in which various embodiments may be implemented;

FIG. 2 shows a schematic embodiment of a client computer;

FIG. 3 illustrates a schematic embodiment of a network computer;

FIG. 4 shows a logical schematic of a system for managing provided patient information, machine learning (ML) models for goals and therapies, analysis, and associated applications;

FIG. 5 illustrates a logical schematic for managing the training of ML models for goals and therapies provided to a patient;

FIG. 6 shows a flowchart for an ML platform that generates and trains goal models and therapy models to provide therapy results that converge with goals;

FIG. 7 illustrates a flowchart for a process for an ML platform that generates patient profiles that are employed to train and retrain models until therapy results converge with goals;

FIG. 8 shows a user interface for selecting patient management applications; and

FIG. 9 shows a user interface for a patient analysis application in accordance with one or more of the various embodiments.

### DESCRIPTION OF VARIOUS EMBODIMENTS

Various embodiments now will be described more fully hereinafter with reference to the accompanying drawings, which form a part hereof, and which show, by way of illustration, specific exemplary embodiments by which the invention may be practiced. The embodiments may, however, be embodied in many different forms and should not be construed as limited to the embodiments set forth herein; rather, these embodiments are provided so that this disclosure will be thorough and complete, and will fully convey the scope of the embodiments to those skilled in the art. Among other things, the various embodiments may be methods, systems, media or devices. Accordingly, the various embodiments may take the form of an entirely hardware embodiment, an entirely software embodiment or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. Also, throughout the specification and the claims, the use of “when” and “responsive to” do not imply that associated resultant actions are required to occur immediately or within a particular time period. Instead they are used herein to indicate actions that may occur or be performed in response to one or more conditions being met, unless the context clearly dictates otherwise. In addition, throughout the specification, the meaning of “a,” “an,” and “the” include plural references. The meaning of “in” includes “in” and “on.”

For example, embodiments, the following terms are also used herein according to the corresponding meaning, unless the context clearly dictates otherwise.

As used herein the term, “engine” refers to logic embodied in hardware or software instructions, which can be written in a programming language, such as C, C++, Objective-C, COBOL, Java™, PHP, Perl, Python, JavaScript, Ruby, VBScript, Microsoft .NET™ languages such as C#, or the like. An engine may be compiled into executable programs or written in interpreted programming languages. Software engines may be callable from other engines or from themselves. Engines described herein refer to one or more logical modules that can be merged with other engines or applications, or can be divided into sub-engines. The engines can be stored in non-transitory computer-readable medium or computer storage device and be stored on and executed by one or more general purpose computers, thus creating a special purpose computer configured to provide the engine.

As used herein, the terms “raw data set,” or “raw data” refer to data sets provided by an organization that may represent the items to be ingested for use in a machine learning repository. In some embodiments raw data may be provided in various formats. In simple cases, raw data may be provided in spreadsheets, databases, csv files, or the like. In other cases, raw data may be provided using structured XML files, tabular formats, JSON files, or the like. In one or more of the various embodiments, raw data in this context may be the product one or more preprocessing operations. For example, one or more pre-processing operations may be executed on information, such as, medical records, patient inquiry forms, log files, data dumps, event logs, database dumps, unstructured data, structured data, or the like, or combination thereof. In some cases, the pre-processing may include data cleansing, filtering, or the like. The particular pre-processing operations may be specialized based on the source, context, format, veracity of the information, or the like. In some cases raw data may include sensitive or confidential information, such as, proprietary information, patient information, or other personally identifiable information.

As used herein, the term “raw data objects” refer to objects that comprise raw datasets. For example, if a raw dataset is comprised of a plurality of tabular record sets, the separate tabular record sets may be considered raw data objects.

As used herein, the term “model object” refers to an object that models various characteristics of an entity or data object. Model objects may include one or more model object fields that represent features or characteristics. Model objects, model object fields, or model object relationship may be governed by a model schema.

As used herein, the term “model schema” refers to a schema that defines model object types, model object features, model object relationships, or the like, that may be supported by the machine learning repository. For example, raw data objects are transformed into model objects that conform to a model schema supported by the machine learning platform.

As used herein, the term “data model” refers to a data structure that represents one or more model objects and their relationships. A data model will conform to a model schema supported by the machine learning platform.

As used herein, the term “parameter model” refers to a data structure that represents one or more model objects that ML models may be arranged to support. A data model that includes model objects may be provided to a ML model if the data model satisfies the requirements of the ML model's parameter model.

As used herein, the terms “machine learning model” or “ML model” refer to machine learning models that may be arranged for scoring or evaluating model objects. The particular type of ML model and the questions it is designed to answer will depend on the application the ML model targets. ML models are associated with parameter models that define model objects that the ML model supports.

As used herein, the terms “machine learning model envelope,” or “ML model envelope” refer to a data structure that includes one or more ML models and a parameter model. A ML model envelope may be arranged to include the modules, code, scripts, programs, or the like, for implementing its one or more included ML models.

As used herein, the term “assessment data” refers to an assessment of an activity or sub-activity (e.g., step) performed by a patient which is created by one or more devices or entities. Assessment data may be created in real-time while one or more entities/devices observe an activity, or post hoc based on previously recorded activities. Assessment data may be unstructured data, such as, text, voice dictation, or the like. Assessment data may include some structured or semi-structured data such as standardized forms or survey responses. Also, in some embodiments, assessment data may be machine generated by one or more apparatuses, therapy devices, or the like, arranged to measure or evaluate activities or sub-activities of a patient. In some cases, assessment data may be correlated with a point in time, such as an amount of time elapsed from the beginning of the activity, such that portions of the assessment data may later be associated with the activity or sub-activity taking place at that point in time.

The following briefly describes the various embodiments to provide a basic understanding of some aspects of the invention. This brief description is not intended as an extensive overview. It is not intended to identify key or critical elements, or to delineate or otherwise narrow the scope. Its purpose is merely to present some concepts in a simplified form as a prelude to the more detailed description that is presented later. A Machine learning models optimize personalized goals and therapies for the patient based on the diagnosis and the received assessment data.

Briefly stated, various embodiments are directed towards a patient management platform for characterizing one or more neuropsychiatric and/or neurological disorders of a patient based on received assessment data, which is employed to generate a diagnosis, goals and therapies for the patient. The platform uses machine learning tools to optimize personalized diagnosis, goals and therapies for the patient based at least on several different types of received assessment data and heuristic data. In one or more of the various embodiments, the patient management platform employs one or more machine learning (ML) engines that may be instantiated to perform one or more portions of the actions described below.

In one or more embodiments, received assessment data for a patient is employed to diagnosis one or more disorders of the patient. Different types of assessment data may include: layperson data, clinical data, biometric data, video data, medical data, heuristic data, or the like. Also, one or more types of the assessment data may be weighted differently, e.g., clinical data may be weighted higher than layperson data. Also, in one or more embodiments, the received assessment data may be provided in a raw data format that is subsequently normalized into data format that may be processed by the machine learning engines.

In one or more embodiments, the one or more patient disorders can include: Autism Spectrum Disorder, attention deficit disorder, oppositional defiant disorder, specific learning disorders, a speech disorder/impairment, attention deficit hyperactive disorder, Tourette's Syndrome, obsessive compulsive disorder, sensory integration disorder, depression, or any other neurological and/or neuropsychiatric disorder.

In one or more embodiments, a profile of the patient is generated based on one or more of a diagnosis, the received assessment information, heuristic data, one or more other patient profiles previously generated for one or more other patients that are associated with one or more similar diagnosis, or the like.

In one or more embodiments, one or more goal models are generated based on the patient's profile. Also, the one or more goal models are trained with one or more of the heuristic data, and the different types of received assessment data to generate one or more goals for the patient.

In one or more embodiments, one or more therapy models are generated based on one or more of the goals, or therapy models previously generated for the one or more other patients that may have a similar diagnosis.

In one or more embodiments, the therapy models are trained with the one or more goals. Also, the trained therapy models are used to generate a treatment plan that includes one or more therapies to be performed with the patient.

In one or more embodiments, in response to the therapy results mismatching the one or more goals, actions are iteratively performed until a match of a goal and a therapy result occurs, such actions includ: (1) retraining the goal models with the mismatched therapy results and the profile; (2) using the retrained goal models to generate retrained goals for the patient; (3) retraining the therapy models with the retrained goals; (4) employing the retrained therapy models to generate retrained therapies for the patient.

In one or more embodiments, when a goal or a retrained goal matches a therapy result, the patient's profile is updated and a report is provided.

In one or more embodiments, applications or apps having a user interface are provided to a user for management of a patient's diagnosis, profile, goals, therapies, and patient result analysis, e.g., a patient profile app, a goal model app, a therapy model app, or a patient analysis app.

In one or more embodiments, providing a therapeutic task to the patient at a user interface for an application on an electronic device. The task is configured to prompt one or more types of behaviors by the patient. Employing the user interface to automatically detect performance of the one or more different types of behaviors by the patient.

In one or more embodiments, performing one or more therapies with the patient comprises one or more of: (1) a first task to prompt a neuro-typical behavior by the patent; (2) a second task to prompt a neuro-atypical behavior by the patient; or (3) a third task to prompt emotional significance by the patient.

One of the benefits of the various embodiments is accurately, effectively, and timely providing a diagnosis of Autism Spectrum Disorder in a patient, which is currently hindered by delays (e.g., bureaucratic delays) in connecting potential Autism Spectrum Disorder patients with practitioners able to provide a diagnosis. In some instances, such delays can approach 9-12 months, during which further progression of the disorder may have occurred, and during which a key treatment opportunity may have passed. In characterizing an Autism Spectrum Disorder diagnosis for a patient, the characterization can include a diagnosis of severity and a type (e.g., Classical Autism, Asperger's Syndrome, Childhood Disintegrative Syndrome, Rett's Disorder, Pervasive Developmental Disorder—Not Otherwise Specified, etc.) within a spectrum of autism disorders.

Furthermore, the diagnosis or characterization of Autism Spectrum Disorder can be performed for subjects of any suitable demographic (e.g., age demographic, ethnicity, gender, socioeconomic demographic, health condition, etc.). For example, the various embodiments can diagnose severity and type of autism spectrum disorder for child or adolescent subjects exhibiting related symptoms, to provide treatment recommendations to such patients, and to monitor such patients during treatment of their disorder(s). Additionally, in one or more of the embodiments, any other suitable neurological, neuropsychiatric or non-neurological disorder can be diagnosed and treated with relevant therapies to meet personalized goals, for any other suitable demographic of patients.

Additionally, the diagnosis, goals, and therapies are not only for clinicians to inform their treatment planning and effectiveness of therapies but also to provide a base line for treatment access, frequency and effectiveness for insurers, or others who need to understanding costs vs effectiveness as they assess the treatment plans submitted to them by clinicians who do not use a similar platform.

### Illustrated Operating Environment

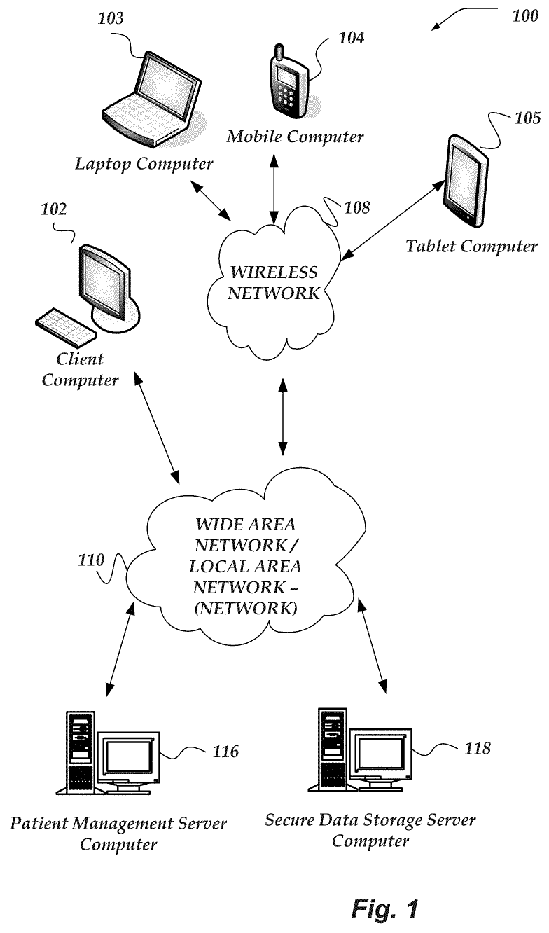

FIG. 1 shows components of one embodiment of an environment in which embodiments of the invention may be practiced. Not all the components may be required to practice the invention, and variations in the arrangement and type of the components may be made without departing from the spirit or scope of the invention. As shown, system 100 of FIG. 1 includes local area networks (LANs)/wide area networks (WANs)—(network) 110, wireless network 108, client computers 102-105, patient management server computer 116, secure data storage server computer 118, or the like.

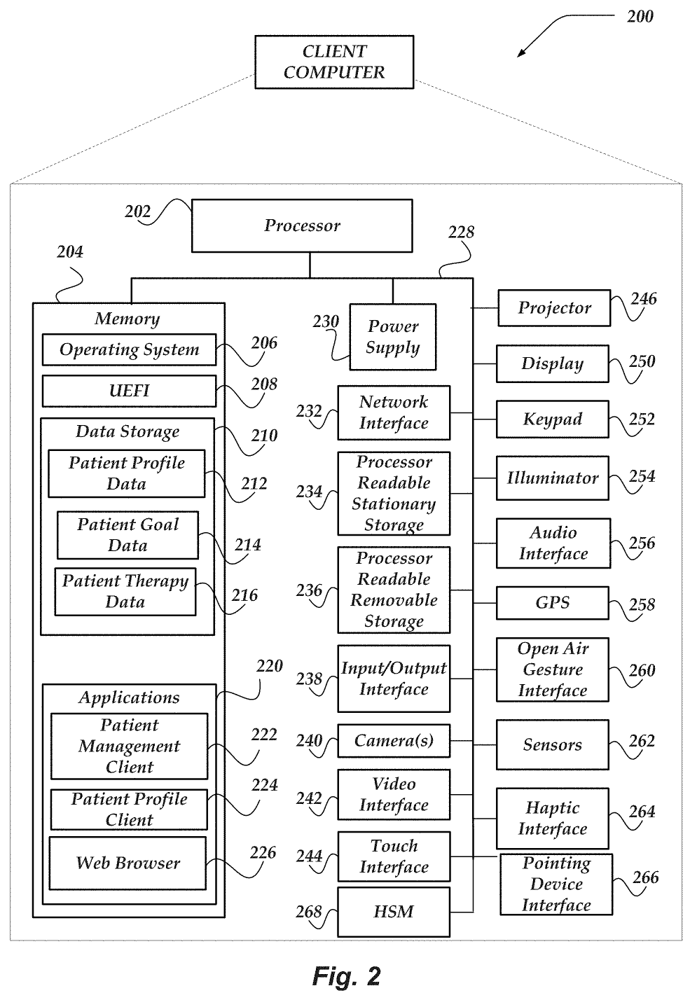

At least one embodiment of client computers 102-105 is described in more detail below in conjunction with FIG. 2. In one embodiment, at least some of client computers 102-105 may operate over one or more wired or wireless networks, such as networks 108, or 110. Generally, client computers 102-105 may include virtually any computer capable of communicating over a network to send and receive information, perform various online activities, offline actions, or the like. In one embodiment, one or more of client computers 102-105 may be configured to operate within a business or other entity to perform a variety of services for the business or other entity. For example, client computers 102-105 may be configured to operate as a web server, firewall, client application, media player, mobile telephone, game console, desktop computer, or the like. However, client computers 102-105 are not constrained to these services and may also be employed, for example, as for end-user computing in other embodiments. It should be recognized that more or less client computers (as shown in FIG. 1) may be included within a system such as described herein, and embodiments are therefore not constrained by the number or type of client computers employed.

Computers that may operate as client computer 102 may include computers that typically connect using a wired or wireless communications medium such as personal computers, multiprocessor systems, microprocessor-based or programmable electronic devices, network PCs, or the like. In some embodiments, client computers 102-105 may include virtually any portable computer capable of connecting to another computer and receiving information such as, laptop computer 103, mobile computer 104, tablet computers 105, or the like. However, portable computers are not so limited and may also include other portable computers such as cellular telephones, display pagers, radio frequency (RF) devices, infrared (IR) devices, Wifi devices, Bluetooth devices, Piconet Devices, Personal Area Network (PAN) devices, Personal Digital Assistants (PDAs), handheld computers, wearable computers, integrated devices combining one or more of the preceding computers, or the like. As such, client computers 102-105 typically range widely in terms of capabilities and features. Moreover, client computers 102-105 may access various computing applications, including a browser, or other web-based application.

A web-enabled client computer may include a browser application that is configured to receive and to send web pages, web-based messages, and the like. The browser application may be configured to receive and display graphics, text, multimedia, and the like, employing virtually any web-based language, including a wireless application protocol messages (WAP), and the like. In one embodiment, the browser application is enabled to employ Handheld Device Markup Language (HDML), Wireless Markup Language (WML), WMLScript, JavaScript, Standard Generalized Markup Language (SGML), HyperText Markup Language (HTML), eXtensible Markup Language (XML), JavaScript Object Notation (JSON), or the like, to display and send a message. In one embodiment, a user of the client computer may employ the browser application to perform various activities over a network (online). However, another application may also be used to perform various online activities.

Client computers 102-105 also may include at least one other client application that is configured to receive or send content between another computer. The client application may include a capability to send or receive content, or the like. The client application may further provide information that identifies itself, including a type, capability, name, and the like. In one embodiment, client computers 102-105 may uniquely identify themselves through any of a variety of mechanisms, including an Internet Protocol (IP) address, a phone number, Mobile Identification Number (MIN), an electronic serial number (ESN), universally unique identifiers (UUIDs), or other device identifiers. Such information may be provided in a network packet, or the like, sent between other client computers, machine learning management server computer 116, server data storage server computer 118, or other computers.

Client computers 102-105 may further be configured to include a client application that enables an end-user to log into an end-user account that may be managed by another computer, such as machine learning management server computer 116, or the like. Such an end-user account, in one non-limiting example, may be configured to enable the end-user to manage one or more online activities, including in one non-limiting example, project management, software development, system administration, data modeling, search activities, social networking activities, browse various websites, communicate with other users, executing one or more healthcare applications in sandbox engines, or the like. Also, client computers may be arranged to enable users to display reports, interactive user-interfaces, or results provided by machine learning management server computer 116 or server data storage server computer 118.

Wireless network 108 is configured to couple client computers 103-105 and its components with network 110. Wireless network 108 may include any of a variety of wireless sub-networks that may further overlay stand-alone ad-hoc networks, and the like, to provide an infrastructure-oriented connection for client computers 103-105. Such sub-networks may include mesh networks, Wireless LAN (WLAN) networks, cellular networks, and the like. In one embodiment, the system may include more than one wireless network.

Wireless network 108 may further include an autonomous system of terminals, gateways, routers, and the like connected by wireless radio links, and the like. These connectors may be configured to move freely and randomly and organize themselves arbitrarily, such that the topology of wireless network 108 may change rapidly.

Wireless network 108 may further employ a plurality of access technologies including 2nd (2G), 3rd (3G), 4th (4G) 5th (5G) generation radio access for cellular systems, WLAN, WiFi, Wireless Router (WR) mesh, and the like. Access technologies such as 2G, 3G, 4G, 5G, and future access networks may enable wide area coverage for mobile computers, such as client computers 103-105 with various degrees of mobility. In one non-limiting example, wireless network 108 may enable a radio connection through a radio network access such as Global System for Mobil communication (GSM), General Packet Radio Services (GPRS), Enhanced Data GSM Environment (EDGE), code division multiple access (CDMA), time division multiple access (TDMA), Wideband Code Division Multiple Access (WCDMA), High Speed Downlink Packet Access (HSDPA), Long Term Evolution (LTE), and the like. In essence, wireless network 108 may include virtually any wireless communication mechanism by which information may travel between client computers 103-105 and another computer, network, a cloud-based network, a cloud instance, or the like.

Network 110 is configured to couple network computers with other computers, including, machine learning management server computer 116, secure data storage server computer 118, client computers 102-105 through wireless network 108, or the like. Network 110 is enabled to employ any form of computer readable media for communicating information from one electronic device to another. Also, network 110 can include the Internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another. In addition, communication links within LANs typically include twisted wire pair or coaxial cable, while communication links between networks may utilize analog telephone lines, full or fractional dedicated digital lines including T1, T2, T3, and T4, or other carrier mechanisms including, for example, E-carriers, Integrated Services Digital Networks (ISDNs), Digital Subscriber Lines (DSLs), wireless links including satellite links, or other communications links known to those skilled in the art. Moreover, communication links may further employ any of a variety of digital signaling technologies, including without limit, for example, DS-0, DS-1, DS-2, DS-3, DS-4, OC-3, OC-12, OC-48, or the like. Furthermore, remote computers and other related electronic devices could be remotely connected to either LANs or WANs via a modem and temporary telephone link. In one embodiment, network 110 may be configured to transport information of an Internet Protocol (IP).

Additionally, communication media typically embodies computer readable instructions, data structures, program modules, or other transport mechanism and includes any information non-transitory delivery media or transitory delivery media. By way of example, communication media includes wired media such as twisted pair, coaxial cable, fiber optics, wave guides, and other wired media and wireless media such as acoustic, RF, infrared, and other wireless media.

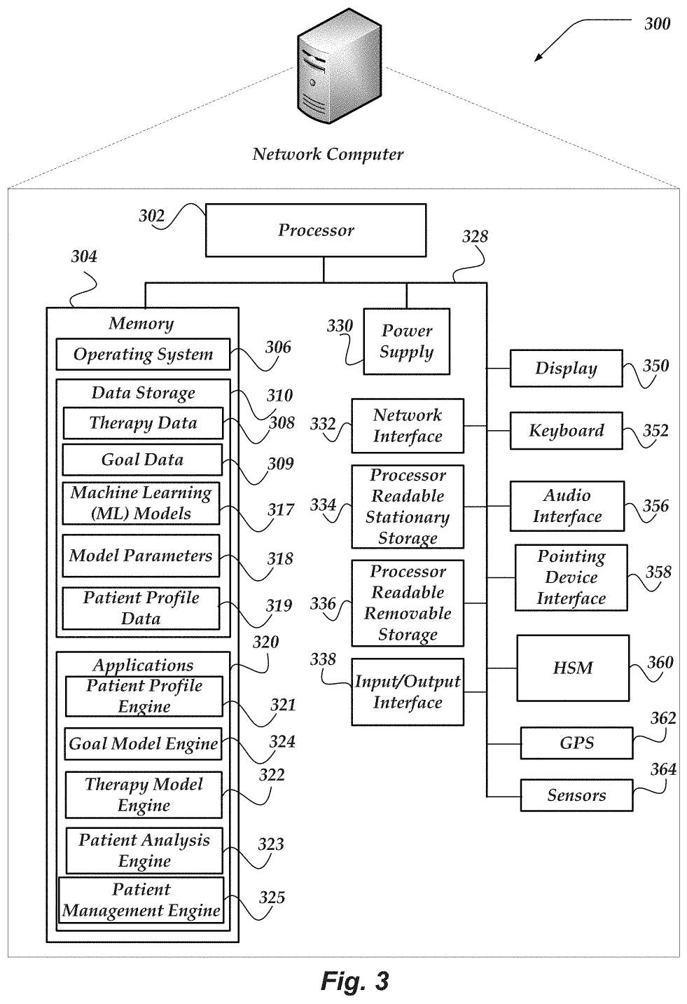

One embodiment of patient management server computer 116 is described in more detail below in conjunction with FIG. 3. Briefly, however, patient management server computer 116 includes virtually any network computer that is specialized to provide data modeling or machine learning services as described herein.

One embodiment of secure data storage server computer 118 is described in more detail below in conjunction with FIG. 3. Briefly, however, secure data storage server computer 118 includes virtually any network computer that is specialized to store user data and machine learning models apart from patient management server computer 116, as described herein.

Although FIG. 1 illustrates patient management server computer 116 and secure data storage server computer 118 as single computers, the innovations or embodiments are not so limited. For example, one or more functions of patient management server computer 116, secure data storage server computer 118, or the like, may be distributed across one or more distinct network computers. Moreover, patient management server computer 116 and secure data storage server computer 118 are not limited to a particular configuration such as the one shown in FIG. 1. Thus, in one embodiment, patient management server computer 116 or secure data storage server computer 118 may be implemented using a plurality of network computers. In other embodiments, server computers may be implemented using a plurality of network computers in a cluster architecture, a peer-to-peer architecture, or the like. Further, in at least one of the various embodiments, patient management server computer 116 or secure data storage server computer 118 may be implemented using one or more cloud instances in one or more cloud networks. Accordingly, these innovations and embodiments are not to be construed as being limited to a single environment, and other configurations, and architectures are also envisaged.

### Illustrative Client Computer

FIG. 2 shows one embodiment of client computer 200 that may include more or less components than those shown. Client computer 200 may represent, for example, at least one embodiment of mobile computers or client computers shown in FIG. 1.

Client computer 200 may include one or more processors, such as processor 202 in communication with memory 204 via bus 228. Client computer 200 may also include power supply 230, network interface 232, audio interface 256, display 250, keypad 252, illuminator 254, video interface 242, input/output interface 238, haptic interface 264, global positioning systems (GPS) receiver 258, open air gesture interface 260, temperature interface 262, camera(s) 240, projector 246, pointing device interface 266, processor-readable stationary storage device 234, and processor-readable removable storage device 236. Client computer 200 may optionally communicate with a base station (not shown), or directly with another computer. And in one embodiment, although not shown, a gyroscope, accelerometer, or the like may be employed within client computer 200 to measuring or maintaining an orientation of client computer 200.

Power supply 230 may provide power to client computer 200. A rechargeable or non-rechargeable battery may be used to provide power. The power may also be provided by an external power source, such as an AC adapter or a powered docking cradle that supplements or recharges the battery.

Network interface 232 includes circuitry for coupling client computer 200 to one or more networks, and is constructed for use with one or more communication protocols and technologies including, but not limited to, protocols and technologies that implement any portion of the OSI model for mobile communication (GSM), CDMA, time division multiple access (TDMA), UDP, TCP/IP, SMS, MMS, GPRS, WAP, UWB, WiMax, WiFi, Bluetooth, SIP/RTP, GPRS, EDGE, WCDMA, LTE, UMTS, OFDM, CDMA2000, EV-DO, HSDPA, 2G, 3G, 4G, 5G or any of a variety of other wireless communication protocols. Network interface 232 is sometimes known as a transceiver, transceiving device, or network interface card (NIC).

Audio interface 256 may be arranged to produce and receive audio signals such as the sound of a human voice. For example, audio interface 256 may be coupled to a speaker and microphone (not shown) to enable telecommunication with others or generate an audio acknowledgement for some action. A microphone in audio interface 256 can also be used for input to or control of client computer 200, e.g., using voice recognition, detecting touch based on sound, and the like.

Display 250 may be a liquid crystal display (LCD), gas plasma, electronic ink, electronic paper, light emitting diode (LED), Organic LED (OLED) or any other type of light reflective or light transmissive display that can be used with a computer. Display 250 may also include a touch interface 244 arranged to receive input from an object such as a stylus or a digit from a human hand, and may use resistive, capacitive, surface acoustic wave (SAW), infrared, radar, or other technologies to sense touch or gestures.

Projector 246 may be a remote handheld projector or an integrated projector that is capable of projecting an image on a remote wall or any other reflective object such as a remote screen.

Video interface 242 may be arranged to capture video images, such as a still photo, a video segment, an infrared video, or the like. For example, video interface 242 may be coupled to a digital video camera, a web-camera, or the like. Video interface 242 may comprise a lens, an image sensor, and other electronics. Image sensors may include a complementary metal-oxide-semiconductor (CMOS) integrated circuit, charge-coupled device (CCD), or any other integrated circuit for sensing light.

Keypad 252 may comprise any input device arranged to receive input from a user. For example, keypad 252 may include a push button numeric dial, or a keyboard. Keypad 252 may also include command buttons that are associated with selecting and sending images.

Illuminator 254 may provide a status indication or provide light. Illuminator 254 may remain active for specific periods of time or in response to events. For example, when illuminator 254 is active, it may backlight the buttons on keypad 252 and stay on while the client computer is powered. Also, illuminator 254 may backlight these buttons in various patterns when particular actions are performed, such as dialing another client computer. Illuminator 254 may also cause light sources positioned within a transparent or translucent case of the client computer to illuminate in response to actions.

Further, client computer 200 may also comprise hardware security module (HSM) 268 for providing additional tamper resistant safeguards for generating, storing or using security/cryptographic information such as, keys, digital certificates, passwords, passphrases, two-factor authentication information, or the like. In some embodiments, hardware security module may be employed to support one or more standard public key infrastructures (PKI), and may be employed to generate, manage, or store keys pairs, or the like. In some embodiments, HSM 268 may be arranged as a hardware card that may be added to a client computer.

Client computer 200 may also comprise input/output interface 238 for communicating with external peripheral devices or other computers such as other client computers and network computers. The peripheral devices may include an audio headset, display screen glasses, remote speaker system, remote speaker and microphone system, and the like. Input/output interface 238 can utilize one or more technologies, such as Universal Serial Bus (USB), Infrared, WiFi, WiMax, Bluetooth™, Bluetooth Low Energy. or the like.

Haptic interface 264 may be arranged to provide tactile feedback to a user of the client computer. For example, the haptic interface 264 may be employed to vibrate client computer 200 in a particular way when another user of a computer is calling. Open air gesture interface 260 may sense physical gestures of a user of client computer 200, for example, by using single or stereo video cameras, radar, a gyroscopic sensor inside a computer held or worn by the user, or the like. Camera 240 may be used to track physical eye movements of a user of client computer 200.

In at least one of the various embodiments, client computer 200 may also include sensors 262 for determining geolocation information (e.g., GPS), monitoring electrical power conditions (e.g., voltage sensors, current sensors, frequency sensors, and so on), monitoring weather (e.g., thermostats, barometers, anemometers, humidity detectors, precipitation scales, or the like), light monitoring, audio monitoring, motion sensors, or the like. Sensors 262 may be one or more hardware sensors that collect or measure data that is external to client computer 200

GPS transceiver 258 can determine the physical coordinates of client computer 200 on the surface of the Earth, which typically outputs a location as latitude and longitude values. GPS transceiver 258 can also employ other geo-positioning mechanisms, including, but not limited to, triangulation, assisted GPS (AGPS), Enhanced Observed Time Difference (E-OTD), Cell Identifier (CI), Service Area Identifier (SAI), Enhanced Timing Advance (ETA), Base Station Subsystem (BSS), or the like, to further determine the physical location of client computer 200 on the surface of the Earth. It is understood that under different conditions, GPS transceiver 258 can determine a physical location for client computer 200. In at least one embodiment, however, client computer 200 may, through other components, provide other information that may be employed to determine a physical location of the client computer, including for example, a Media Access Control (MAC) address, IP address, and the like.

In at least one of the various embodiments, applications, such as, machine learning platform client application 222, web browser 226, or the like, may be arranged to employ geo-location information to select one or more localization features, such as, time zones, languages, currencies, calendar formatting, or the like. Localization features may be used in user-interfaces, reports, as well as internal processes or databases. In at least one of the various embodiments, geo-location information used for selecting localization information may be provided by GPS 258. Also, in some embodiments, geolocation information may include information provided using one or more geolocation protocols over the networks, such as, wireless network 108 or network 111.

Human interface components can be peripheral devices that are physically separate from client computer 200, allowing for remote input or output to client computer 200. For example, information routed as described here through human interface components such as display 250 or keyboard 252 can instead be routed through network interface 232 to appropriate human interface components located remotely. Examples of human interface peripheral components that may be remote include, but are not limited to, audio devices, pointing devices, keypads, displays, cameras, projectors, and the like. These peripheral components may communicate over a Pico Network such as Bluetooth™, Zigbee™, Bluetooth Low Energy, or the like. One non-limiting example of a client computer with such peripheral human interface components is a wearable computer, which might include a remote pico projector along with one or more cameras that remotely communicate with a separately located client computer to sense a user's gestures toward portions of an image projected by the pico projector onto a reflected surface such as a wall or the user's hand.

A client computer may include web browser application 226 that may be configured to receive and to send web pages, web-based messages, graphics, text, multimedia, and the like. The client computer's browser application may employ virtually any programming language, including a wireless application protocol messages (WAP), and the like. In at least one embodiment, the browser application is enabled to employ Handheld Device Markup Language (HDML), Wireless Markup Language (WML), WMLScript, JavaScript, Standard Generalized Markup Language (SGML), HyperText Markup Language (HTML), eXtensible Markup Language (XML), HTML5, and the like.

Memory 204 may include RAM, ROM, or other types of memory. Memory 204 illustrates an example of computer-readable storage media (devices) for storage of information such as computer-readable instructions, data structures, program modules or other data. Memory 204 may store Unified Extensible Firmware Interface (UEFI) 208 for controlling low-level operation of client computer 200. The memory may also store operating system 206 for controlling the operation of client computer 200. It will be appreciated that this component may include a general-purpose operating system such as a version of UNIX, or LINUX™, or a specialized client computer communication operating system such as Windows Phone™. The operating system may include, or interface with a Java or JavaScript virtual machine modules that enable control of hardware components or operating system operations via Java application programs or JavaScript programs.

Memory 204 may further include one or more data storage 210, which can be utilized by client computer 200 to store, among other things, applications 220 or other data. For example, data storage 210 may also be employed to store information that describes various capabilities of client computer 200. The information may then be provided to another device or computer based on any of a variety of events, including being sent as part of a header during a communication, sent upon request, or the like. Data storage 210 may also be employed to store social networking information including address books, buddy lists, aliases, user profile information, user credentials, or the like. Data storage 210 may further include program code, data, algorithms, and the like, for use by a processor, such as processor 202 to execute and perform actions. Program code and data may include patient profile data 212, patient goal data 214, patient therapy data 216. The different types of data may include raw data objects stored in secure on-premise servers or secure cloud servers. The raw data objects may be retrieved from either location and stored in a local state store, or when the amount of raw data exceeds a defined threshold, retrieved via a remote state store proxy.

In one embodiment, at least some of data storage 210 might also be stored on another component of client computer 200, including, but not limited to, non-transitory processor-readable removable storage device 236, processor-readable stationary storage device 234, or even external to the client computer.

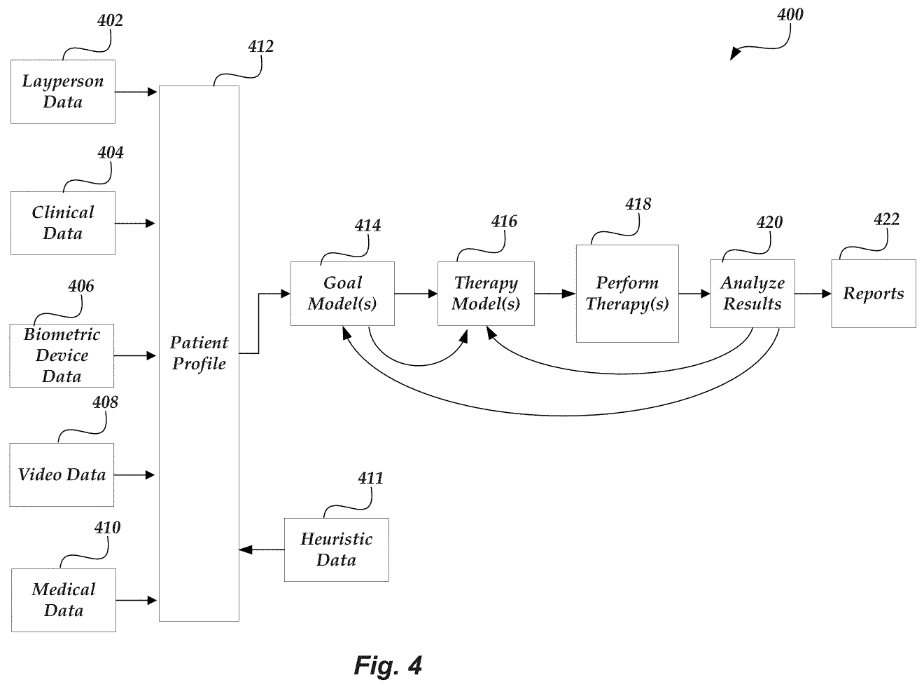

One embodiment of application bundle 212 is described in more detail below in conjunction with FIG. 4. Briefly, however, application bundle 212 comprises one or more applications, such as machine learning based applications. In some embodiments, application bundle 212 includes one or more one or more healthcare applications for execution by a sandbox engine in the context of sandbox 214.

Applications 220 may include computer executable instructions which, when executed by client computer 200, transmit, receive, or otherwise process instructions and data. Applications 220 may include, for example patient management client application 222, patient profile client 224, web browser 226, or the like. In at least one of the various embodiments, patient management client application 222 may be used to interact with a machine learning management server computer, such as patient management server computer 116. Also, patient management client application 222 and patient profile client 224 may provide machine learning functionality.

Other examples of application programs include calendars, search programs, email client applications, IM applications, SMS applications, Voice Over Internet Protocol (VOIP) applications, contact managers, task managers, transcoders, database programs, word processing programs, security applications, spreadsheet programs, games, search programs, and so forth.

Additionally, in one or more embodiments (not shown in the figures), client computer 200 may include one or more embedded logic hardware devices instead of one or more CPUs, such as, an Application Specific Integrated Circuits (ASICs), Field Programmable Gate Arrays (FPGAs), Programmable Array Logic (PAL), or the like, or combination thereof. The embedded logic hardware devices may directly execute embedded logic to perform actions. Also, in one or more embodiments (not shown in the figures), the client computer may include one or more hardware microcontrollers instead of one or more CPUs. In at least one embodiment, the microcontrollers be system-on-a-chips (SOCs) that may directly execute their own embedded logic to perform actions and access their own internal memory and their own external Input and Output Interfaces (e.g., hardware pins or wireless transceivers) to perform actions.

### Illustrative Network Computer

FIG. 3 shows one embodiment of network computer 300 that may be included in a system implementing one or more embodiments of the described innovations. Network computer 300 may include more or less components than those shown in FIG. 3. However, the components shown are sufficient to disclose an illustrative embodiment for practicing these innovations. Network computer 300 may represent, for example, one embodiment of patient management server computer 116 of FIG. 1.

As shown in the figure, network computer 300 includes a processor 302 in communication with a memory 304 via a bus 328. Network computer 300 also includes a power supply 330, network interface 332, audio interface 356, global positioning systems (GPS) receiver 362, display 350, keyboard 352, input/output interface 338, processor-readable stationary storage device 334, and processor-readable removable storage device 336. Power supply 330 provides power to network computer 300. In some embodiments, processor 302 may be a multiprocessor system that includes one or more processors each having one or more processing/execution cores.

Network interface 332 includes circuitry for coupling network computer 300 to one or more networks, and is constructed for use with one or more communication protocols and technologies including, but not limited to, protocols and technologies that implement any portion of the Open Systems Interconnection model (OSI model), global system for mobile communication (GSM), code division multiple access (CDMA), time division multiple access (TDMA), user datagram protocol (UDP), transmission control protocol/Internet protocol (TCP/IP), Short Message Service (SMS), Multimedia Messaging Service (MMS), general packet radio service (GPRS), WAP, ultra wide band (UWB), IEEE 802.16 Worldwide Interoperability for Microwave Access (WiMax), Session Initiation Protocol/Real-time Transport Protocol (SIP/RTP), or any of a variety of other wired and wireless communication protocols. Network interface 332 is sometimes known as a transceiver, transceiving device, or network interface card (NIC). Network computer 300 may optionally communicate with a base station (not shown), or directly with another computer.

Audio interface 356 is arranged to produce and receive audio signals such as the sound of a human voice. For example, audio interface 356 may be coupled to a speaker and microphone (not shown) to enable telecommunication with others or generate an audio acknowledgement for some action. A microphone in audio interface 356 can also be used for input to or control of network computer 300, for example, using voice recognition.

Display 350 may be a liquid crystal display (LCD), gas plasma, electronic ink, light emitting diode (LED), Organic LED (OLED) or any other type of light reflective or light transmissive display that can be used with a computer. Display 350 may be a handheld projector or pico projector capable of projecting an image on a wall or other object.

Network computer 300 may also comprise input/output interface 338 for communicating with external devices or computers not shown in FIG. 3. Input/output interface 338 can utilize one or more wired or wireless communication technologies, such as USB™, Firewire™, WiFi, WiMax, Thunderbolt™, Infrared, Bluetooth™, Zigbee™, serial port, parallel port, and the like.

GPS transceiver 362 can determine the physical coordinates of network computer 300 on the surface of the Earth, which typically outputs a location as latitude and longitude values. GPS transceiver 362 can also employ other geo-positioning mechanisms, including, but not limited to, triangulation, assisted GPS (AGPS), Enhanced Observed Time Difference (E-OTD), Cell Identifier (CI), Service Area Identifier (SAI), Enhanced Timing Advance (ETA), Base Station Subsystem (BSS), or the like, to further determine the physical location of network computer 300 on the surface of the Earth. It is understood that under different conditions, GPS transceiver 362 can determine a physical location for network computer 300.

Network computer 300 may also include sensors 364 for determining geolocation information (e.g., GPS), monitoring electrical power conditions (e.g., voltage sensors, current sensors, frequency sensors, and so on), monitoring weather (e.g., thermostats, barometers, anemometers, humidity detectors, precipitation scales, or the like), light monitoring, audio monitoring, motion sensors, or the like. Sensors 364 may be one or more hardware sensors that collect or measure data that is external to network computer 300

In at least one embodiment, however, network computer 300 may, through other components, provide other information that may be employed to determine a physical location of the client computer, including for example, a Media Access Control (MAC) address, IP address, and the like.

Human interface components can be physically separate from network computer 300, allowing for remote input or output to network computer 300. For example, information routed as described here through human interface components such as display 350 or keyboard 352 can instead be routed through the network interface 332 to appropriate human interface components located elsewhere on the network. Human interface components include any component that allows the computer to take input from, or send output to, a human user of a computer. Accordingly, pointing devices such as mice, styluses, track balls, or the like, may communicate through pointing device interface 358 to receive user input.

Memory 304 may include Random Access Memory (RAM), Read-Only Memory (ROM), or other types of non-transitory computer readable or writeable media. Memory 304 illustrates an example of computer-readable storage media (devices) for storage of information such as computer-readable instructions, data structures, program modules or other data. Memory 304 stores a unified extensible firmware interface (UEFI) 308 for controlling low-level operation of network computer 300. The memory also stores an operating system 306 for controlling the operation of network computer 300. It will be appreciated that this component may include a general-purpose operating system such as a version of UNIX, or LINUX™, or a specialized operating system such as Microsoft Corporation's Windows ® operating system, or the Apple Corporation's OSX® operating system. The operating system may include, or interface with a Java virtual machine module that enables control of hardware components or operating system operations via Java application programs. Likewise, other runtime environments may be included.

Memory 304 may further include one or more data storage 310, which can be utilized by network computer 300 to store, among other things, applications 320 or other data. For example, data storage 310 may also be employed to store information that describes various capabilities of network computer 300. The information may then be provided to another device or computer based on any of a variety of events, including being sent as part of a header during a communication, sent upon request, or the like. Data storage 310 may also be employed to store social networking information including address books, buddy lists, aliases, user profile information, or the like. Data storage 310 may further include program code, data, algorithms, and the like, for use by one or more processors, such as processor 302 to execute and perform actions such as those actions described below. In one embodiment, at least some of data storage 310 might also be stored on another component of network computer 300, including, but not limited to, non-transitory media inside processor-readable removable storage device 336, processor-readable stationary storage device 334, or any other computer-readable storage device within network computer 300, or even external to network computer 300. Data storage 310 may include, for example, therapy data 308, goal data 309, machine learning modles 317, model parameters 318, patient profile data 319, or the like.

Applications 320 may include computer executable instructions which, when executed by network computer 300, transmit, receive, or otherwise process messages (e.g., SMS, Multimedia Messaging Service (MMS), Instant Message (IM), email, or other messages), audio, video, and enable telecommunication with another user of another mobile computer. Other examples of application programs include calendars, search programs, email client applications, IM applications, SMS applications, Voice Over Internet Protocol (VOIP) applications, contact managers, task managers, transcoders, database programs, word processing programs, security applications, spreadsheet programs, games, search programs, and so forth. Applications 320 may include patient profile engine 321, goal model engine 324, therapy model engine 322, patient analysis engine 323, patient management engine 325, or the like, that may perform actions further described below. In at least one of the various embodiments, one or more of the applications may be implemented as modules or components of another application. Further, in at least one of the various embodiments, applications may be implemented as operating system extensions, modules, plugins, or the like.

In at least one of the various embodiments, applications 320, or the like, may be arranged to employ geo-location information to select one or more localization features, such as, time zones, languages, currencies, calendar formatting, or the like. Localization features may be used in user-interfaces, reports, as well as internal processes or databases. In at least one of the various embodiments, geo-location information used for selecting localization information may be provided by GPS 362. Also, in some embodiments, geolocation information may include information provided using one or more geolocation protocols over the networks, such as, wireless network 108 or network 110.

Furthermore, in at least one of the various embodiments, one or more of applications 320, may be operative in a cloud-based computing environment. In at least one of the various embodiments, these engines, and others, may be executing within virtual machines or virtual servers that may be managed in a cloud-based based computing environment. In at least one of the various embodiments, in this context applications including the engines may flow from one physical network computer within the cloud-based environment to another depending on performance and scaling considerations automatically managed by the cloud computing environment. Likewise, in at least one of the various embodiments, virtual machines or virtual servers dedicated to one or more of applications 320, or the like, may be provisioned and de-commissioned automatically.

Further, in some embodiments, network computer 300 may also include hardware security module (HSM) 360 for providing additional tamper resistant safeguards for generating, storing or using security/cryptographic information such as, keys, digital certificates, passwords, passphrases, two-factor authentication information, or the like. In some embodiments, hardware security module may be employed to support one or more standard public key infrastructures (PKI), and may be employed to generate, manage, or store keys pairs, or the like. In some embodiments, HSM 360 may be arranged as a hardware card that may be installed in a network computer.

Additionally, in one or more embodiments (not shown in the figures), network computer 300 may include an one or more embedded logic hardware devices instead of one or more CPUs, such as, Application Specific Integrated Circuits (ASICs), Field Programmable Gate Arrays (FPGAs), Programmable Array Logic (PALs), or the like, or combination thereof. The one or more embedded logic hardware devices may directly execute its embedded logic to perform actions. Also, in one or more embodiments (not shown in the figures), the network computer may include one or more hardware microcontrollers instead of one or more CPUs. In at least one embodiment, the one or more microcontrollers may directly execute embedded logic to perform actions and access their own internal memory and their own external Input and Output Interfaces (e.g., hardware pins or wireless transceivers) to perform actions. E.g., they may be arranged as Systems On Chips (SOCs).

### Illustrative Logical System Architecture

FIG. 4 shows a logical schematic of a portion of patient management system 400 for managing the intake of assessment data to provide a diagnosis and a profile of a patient. In one or more embodiments, the received assessment data includes layperson data 402, clinical assessment data, 404, biometric device assessment data 406, video assessment data 408, medical assessment data 410, and heuristic assessment data 411. Additionally, one or more different weights may be associated with one or more different types of the received assessment data. The available/received different types of assessment data and their associated weights are employed to generate a diagnosis of one or more neuropsychiatric and/or neurological disorders that the patient.

In one or more other embodiments, in addition to the received types of assessment data, the diagnosis included in the profile may also be based on heuristic data for one or more previous diagnosis and/or patient profiles provided for other patients that are associated with previously received assessment data for the other patients that is at least somewhat similar, equivalent or matching within one or more thresholds provided for one or more of the received assessment data, diagnosis, or diagnosis models for the patient.

In one or more other embodiments, in addition to the received types of assessment data, and/or the somewhat similar heuristic data for other patients, the one or more diagnosis may also be based on heuristic data for previously received assessment data for the patient and one or more diagnosis models that are associated with the previously received assessment data for the patient. The one or more diagnosis models may be trained with the received assessment data and one or more portion of the previously received assessment data to generate the one or more diagnosis for the patient.

In one or more embodiments, the patient profile may be based on one or more of the received assessment data, heuristic data, previously provided patient profiles for other patients, or a patient profile model that is associated with and trained with received assessment data to generate a patient profile. In one or more embodiments, the patient profile may include one or more of a diagnosis of a disorder, received assessment data, heuristic data for other diagnosis and/or patient profiles for other patients, or one or more patient profile models.

In one or more embodiments, layperson data 402 regarding neuropsychiatric and/or neurological disorders regarding a patient may be provided by a person (layperson) that is not formally trained or qualified to assess disorders of the patient. One or more laypersons providing such assessment data may include a parent, a relative, a home care assistant, or any other person, that has observed and/or interacted with the patient in a non-clinical and uncontrolled environment.

Further, in one or more embodiments, clinical data 404 may be provided by one or more professional persons that are educated and/or trained to provide assessment data of neuropsychiatric and/or neurological disorders of the patient, which may occur in a clinical and/or controlled environment. The one or more professional persons may include one or more of an occupational therapist, a behavior technician, a speech therapist, a behavioral health provider, a medical doctor, a nurse, or any other person that is formally trained to provide assessment data of a patient with neuropsychiatric and/or neurological disorders in a clinical environment.

In one or more embodiments, the clinical assessment data 404 may include an interview dataset based upon a first set of behaviors of the patient, and functions to receive a survey of information reported regarding the patient that can be used to characterize the state of the disorder for the patient. Preferably, the interview dataset is generated based upon responses to a set of items of a survey, wherein the responses are provided by an entity who has directly or indirectly observed the first set of behaviors of the patient; however, the interview dataset can additionally or alternatively be generated in any other suitable manner. In variations, the entity can be any one or more of: a parent, a sibling, a healthcare provider, a layperson, a professional person, a supervisor, a peer, and any other suitable entity able to accurately provide responses to the set of items of the survey. Furthermore, the survey is preferably provided to the entity electronically (e.g., at a mobile application, at a web application, using a messaging client, using an email client, etc.); however, the survey can additionally or alternatively be provided to the entity non-electronically (e.g., by paper, verbally, etc.).

In one or more embodiments, the survey can be provided to the entity in modules, wherein the modules are provided upon one or more triggers (e.g., a behavior of the subject can trigger provision of a module of the survey), at regular or irregular intervals of time (e.g., at certain ages or developmental stages of the subject), and/or in any other suitable manner. Additionally, or alternatively, the survey can be provided to the entity in completion. For example, in one or more embodiments, for characterization of Autism Spectrum Disorder in a patient, the interview dataset can be derived from a survey comprising content of the Autism Diagnostic Interview-Revised (ADI-R). As such, the interview dataset can include responses to 93 items (or any other suitable number of items) of the ADI-R survey divided into three behavioral areas including 1) social interaction, 2) communication and language, and 3) restricted and repetitive behaviors.

However, in other embodiments for Autism Spectrum Disorder characterization and diagnosis, the interview dataset can additionally or alternatively include responses to a survey comprising content derived from any one or more of: the Autism Diagnostic Interview (ADI), the Social Communication Questionnaire (SCQ), and any other suitable instrument configured to facilitate documentation of behaviors indicative or not indicative of Autism Spectrum Disorder. Alternatively, the interview dataset can additionally or alternatively be derived from any other suitable instrument, survey, and/or diagnostic manual (e.g., a version of the Diagnostic and Statistical Manual of Mental Disorders) configured to characterize a state of any other suitable disorder.

In one or more embodiments, for characterization of an Autism Spectrum Disorder state in a patient, the first set of behaviors preferably includes behaviors related to any one or more of: communication and language skills (e.g., speech development, appropriate word use, ability to sustain a conversation, etc.), social interaction issues (e.g., emotional response interpretation, display of emotional responses, irregularities in focus, irregularities in making eye contact, etc.), repetitive and obsessive behaviors (e.g., fixation on items, repetition of words or phrases out of context, repetitive motions such as flapping or pacing, etc.), ability to perform tasks (e.g., pointing, showing) when prompted, and any other suitable behavior indicative or not indicative of a state of Autism Spectrum Disorder. The first set of behaviors can include behaviors exhibited or not exhibited currently by the subject, and can additionally or alternatively include behaviors exhibited or not exhibited by the subject at a past time point (e.g., when the subject was at a given age or within a range of ages prior to the present). In these variations for Autism Spectrum Disorder, the first set of behaviors is preferably observed and captured in the interview dataset for a subject greater than 18 months of age; however, the first set of behaviors can additionally or alternatively be determined for a subject of any suitable age demographic. As such, responses to the survey contribute to the interview dataset, which can identify whether the subject exhibits behaviors indicative or not indicative of a state of Autism Spectrum Disorder, based upon observation of the first set of behaviors by an overseeing entity.

In one or more embodiments, variations of the method for characterizing and diagnosing a non-Autism Spectrum Disorder, may employ any other suitable dataset based upon any other suitable set of behaviors or factors (e.g., biometric, genetic, etc.) of the subject.

However, the interview dataset preferably includes a quantified score for the response(s) to each item and/or group of items of a survey, which can be processed and/or reduced to determine a metric. The quantified score(s) for each item or group of items can be generated from a set of qualitative criteria, wherein each qualitative criterion of the set of qualitative criteria is mapped to a quantified metric (e.g., a number along a scale). However, the quantified score(s) for each item or group of items can additionally or alternatively be generated in any other suitable manner. For instance, the number of instances in which a subject exhibits a behavior (e.g., total number, number within a given time period, difference in number between different time points or time periods, etc.) can be used to generate a quantified score for an item of the survey. In a specific example case for Autism Spectrum Disorder characterization according to the ADI-R, responses to each of 93 items (or any other suitable number of items) can be scored on a scale from zero to nine, wherein a score of zero indicates that a “behavior of the type specified in the coding is not present”, a score of one indicates that a “behavior of the type specified is present in an abnormal form, but not sufficiently severe or frequent to meet the criteria for a 2”, a score of 2 indicates “definite abnormal behavior”, a score of 3 indicates “extreme severity of the specified behavior”, a score of 7 indicates “definite abnormality in the general area of the coding, but not of the type specified”, a score of 8 indicates “not applicable”, and a score of 9 indicates “not known or asked”.

With regard to the ADI-R, scores from individual items are aggregated to generate scores for each of three behavioral areas (e.g., by one or more of averaging, adding, weighting, and subtracting scores), which can be used to determine a metric that may vary. These variations can, however, include generation and/or aggregation of quantified scores from the first set of behaviors, in any other suitable manner. Alternatively, generation of the interview dataset may not include generation of one or more quantified scores.

Also, in one or more embodiments, biometric device data 406 may be provided by one or more physical biometric devices employed to measure one or more biological signals or physical actions of the patient to provide assessment data of neuropsychiatric and/or neurological disorders of the patient, which may occur in a clinical or non-clinical environment. The measured signals or actions may include one or more of: heart rate, skin temperature, limb movements, galvanic skin resistance, ambient heat, ambient noise, ambient light, or the like. The measured signal or actions may be compared to a profile to identify triggering incidents and/or patterns so that severe incidents of anxiety and/or seizures of the patient may be reduced or ameliorated with one or more personalized goals and therapies.

In one or more embodiments, medical data 410 may be provided by one or more professional personas such as medical care providers, naturopathic care providers, wellness providers, and the like. Medical data 410 can include diagnosis of various conditions associated with the patient, and physical information. For example, one or more of gender, weight, height, mobility, deafness, blindness, retardation, birth defects, disease, dementia, or the like

In one or more embodiments, video data 408 may be provided by one or more cameras that are employed to measure one or more physical actions, facial expressions, eye movement, or the like, of the patient to provide video assessment data of one or more neuropsychiatric and/or neurological disorders. The video data may also be employed to measure one or more of ambient lighting, ambient activity, or ambient noise in a clinical or non-clinical environment. The measured actions and environmental measurements may be compared to a profile to identify triggering incidents and/or patterns so that severe incidents of anxiety and/or seizures of the patient may be reduced or ameliorated with treating the patient with one or more of the therapies.

In one or more embodiments, an observation dataset may be based upon a video dataset capturing a second set of behaviors of the patient, and functions to receive an additional set of information regarding the patient that can be used to characterize the state of the disorder for the patient. In variations for characterization of an Autism Spectrum Disorder state, the observation dataset and/or the video dataset are preferably generated according to methods derived from the

Autism Diagnostic Observation Schedule (ADOS) or a variation thereof (e.g., ADOS-2, ADOS-G, etc.) and can additionally or alternatively include annotated methods of the ADOS and/or items not included in the ADOS. Alternatively, the observation dataset and/or the video dataset can be generated according to any other suitable instrument for characterization of Autism Spectrum Disorder or any other suitable disorder based upon observation of behaviors. The video dataset is preferably captured in real time; however, the video dataset can alternatively be captured in non-real time. Furthermore, the video dataset preferably includes a set of video clips, taken at different time points; however, the video dataset can alternatively be a continuous video stream spanning a duration of time without any breaks. In variations wherein the video dataset includes a set of video clips, each video clip in the set of video clips can span any suitable duration of time and/or can be received in real or non-real time. Furthermore, capture of each video clip can be triggered automatically (e.g., based upon sensor detection of a behavior) or performed manually.

Preferably, the video dataset is generated by guiding an entity in communication with the patient to capture the video dataset, wherein the entity can be the same entity, or a different entity. In variations, the entity can be any one or more of: a parent, a sibling, a healthcare provider, a supervisor, a peer, a layperson, and any other suitable entity able to accurately provide responses to the set of items of the survey. Furthermore, guidance of the entity in capturing the video dataset is preferably performed by providing the entity with a set of instructions electronically (e.g., at a mobile application, at a web application, using a messaging client, using an email client, using audio, using video, etc.) at a user interface of a device able to capture the video dataset; however, the set of instructions can additionally or alternatively be provided to the entity non-electronically or electronically (e.g., by paper, verbally, visually, etc.) at an interface separate from that of a device able to capture the video dataset. The instructions preferably guide the entity in administering tasks or activities to the patient, in order to prompt at least one behavior, but can additionally or alternatively guide the entity in passively capturing behaviors of the patient. However, the video dataset can additionally or alternatively be generated in any other suitable manner (e.g., based upon automatic capture, based upon capture by a non-human entity, etc.).

In variations of characterization of an Autism Spectrum Disorder state in a patient, the second set of behaviors preferably includes behaviors related to any one or more of: behaviors prior to and post development of motor coordination skills (e.g., walking), behaviors prior to competency in using phrase speech, behaviors post usage of phrase speech but prior to language fluency, behaviors post language fluency, pre-adolescent behaviors, and post-adolescent behaviors. Also, the second set of behaviors can additionally or alternatively include behaviors related to one or more of: communication and language skills (e.g., speech development, appropriate word use, ability to sustain a conversation, etc.), social interaction issues (e.g., emotional response interpretation, display of emotional responses, irregularities in focus, irregularities in making eye contact, etc.), repetitive and obsessive behaviors (e.g., fixation on items, repetition of words or phrases out of context, repetitive motions such as flapping or pacing, etc.), ability to perform tasks (e.g., pointing, showing) when prompted, and any other suitable behavior indicative or not indicative of a state of Autism Spectrum Disorder. In these variations for Autism Spectrum Disorder, the second set of behaviors is preferably observed and captured in the video dataset for a patient who exhibits some motor coordination (e.g., walking); however, the second set of behaviors can additionally or alternatively be captured for a patient of any suitable age or developmental stage demographic.

The observation dataset is generated based upon the video dataset, and preferably includes documentation of the second set of behaviors captured in the video dataset. As such, generation of the observation dataset can include manual processing, semi-automatic processing, and/or automatic processing of the video dataset to extract behaviors of the second set of behaviors indicative of a disorder state or not indicative of a disorder state (e.g., according to the ADOS, according to the ADOS-2, according to any suitable instrument, etc.). Manual or semi-automatic processing of the video dataset can include identifying behaviors indicative of the disorder state or not indicative of the disorder state by an analyst (e.g., human analyst) examining the video dataset.

In one or more embodiments, semi-automatic or automatic processing of the video dataset can include automatic identification of behaviors indicative of the disorder state or not indicative of the disorder state by a processor analyzing the video dataset, wherein the processor implements a visual detection algorithm for identifying one or more behaviors. In some variations of semi-automatic or automatic processing, the visual detection algorithm can implement machine learning algorithms that improve detection of such behaviors based upon acquisition of additional data and/or implementation of a training dataset (e.g., a set of data including captured and identified behaviors to train the machine learning algorithms). The observation dataset can thus be generated in near-real time upon reception of the video dataset, or in non-real time. As such, transformation of the video dataset into an observation dataset helps identify whether the patient exhibits behaviors indicative or not indicative of a disorder state (e.g., a state of Autism Spectrum Disorder), based upon capture of the second set of behaviors in the video dataset. Also, embodiments for characterizing non-autism disorders, can include receiving any other suitable dataset based upon any other suitable set of behaviors or factors (e.g., biometric, genetic, etc.) of the patient.

The observation dataset preferably includes a quantified score for at least one behavior of the second set of behaviors captured in the video dataset, which can be processed and/or reduced to determine a metric. The quantified score(s) for the captured behavior(s) can be generated from a set of qualitative criteria, wherein each qualitative criterion of the set of qualitative criteria is mapped to a quantified metric (e.g., a number along a scale). However, the quantified score(s) for each item or group of items can additionally or alternatively be generated in any other suitable manner. For instance, the number of instances in which a patient exhibits a behavior (e.g., total number, number within a given time period, difference in number between different time points or time periods, etc.), as captured in the video dataset, can be used to generate a quantified score for the observed behavior. The quantified score(s) can be generated by an entity, a trained analyst, a professional person, a processor, and/or any other suitable entity. In a specific example case for Autism Spectrum Disorder characterization according to the ADOS, scoring of behaviors according to modules targeted to different stages of development (e.g., motor skill development, speech development, etc.) can including mapping of an “intensity” of a behavior to a quantified score. Furthermore, with regard to the ADOS, quantified scores from each behavior and/or module can be aggregated to generate one or more aggregate scores, to determine a metric. Variations of the specific example can, however, include generation and/or aggregation of quantified scores from the second set of behaviors, in any other suitable manner. Alternatively, generation of the observation dataset may not include generation of one or more quantified scores.

In other embodiments, any other suitable instruments for characterizing or diagnosing a disorder, or instruments derived from these instruments, can be implemented with respect to the patient to generate suitable datasets. Furthermore, the datasets can be overlapping, which allows for verification of behaviors reported or captured in different manners. As such, overlapping datasets can facilitate authentication (e.g., by redundancy) of a reported behavior, identification of contractions between reported and observed behaviors, and/or can be used for any other suitable purpose. For instance, an entity-reported behavior according to a survey may be verified by a behavior captured in a video dataset. Additionally, or alternatively, at least some portions of multiple datasets can be complementary, in order to characterize a more complete set of behaviors exhibited by the patient. For example, some entity-reported behaviors may be difficult to capture in a video dataset, and some behaviors capturable in a video dataset may not be easily recognized or reported by an entity.

In one or more embodiments, an aggregate reduced dataset may be generated based upon a reduction of at least one of the interview dataset and the observation dataset, and functions to reduce redundancy in and/or increase the efficiency of acquisition of the interview dataset and the observation dataset. Generating the aggregate reduced dataset can be performed prior to, subsequent to, or simultaneously with reduction of at least one of the interview dataset and the observation dataset.

In one or more embodiments, the interview dataset and the observation dataset can be aggregated prior to reduction, wherein aggregation includes grouping quantified scores of the interview dataset and the observation dataset by behavior category. In an example for Autism Spectrum Disorder characterization, all scores for behaviors of the first and the second set of behaviors related to social interaction can be grouped in a first category, all scores for behaviors of the first and the second set of behaviors related to communication and language can be grouped in a second category, and all scores for behaviors of the first and the second set of behaviors related to restricted and repetitive behaviors can be grouped in a third category, thus aggregating the interview and the observation datasets, and organizing the aggregate dataset into groups. However, in variations of the first variation, aggregation can be performed in any other suitable manner (e.g., with or without grouping).

After aggregation of the interview and the observation datasets in the first variation, the aggregate dataset can be reduced according to any suitable algorithm to account for redundancy, contradictions, and/or any other suitable artifact of the aggregate dataset. As such, reducing can include any one or more of: omitting scores based upon an identified redundancy, weighting scores based upon an identified redundancy (e.g., one or more scores for redundant items from the interview dataset and the observation dataset can be given a lower weight in generation of the aggregate reduced dataset), weighting scores based upon an identified importance (e.g., one or more scores for important items from the interview dataset and the observation dataset can be given a higher weight in generation of the aggregate reduced dataset), adding scores based upon an identified importance (e.g., one or more scores for important items from the interview dataset and the observation dataset can be added in generation of the aggregate reduced dataset), subtracting scores based upon an identified importance (e.g., scores from important items in the interview dataset and the observation dataset can be subtracted from each other in generation of the aggregate reduced dataset), averaging scores (e.g., determining a mean, a median, a mode) based upon an identified importance (e.g., multiple scores can be averaged in generation of the aggregate reduced dataset), and any other suitable mathematical operation that can be performed for scores from the aggregate dataset.

In one or more embodiments, importance can be determined based upon a finding of efficacy or non-efficacy in accurately determining a state of the disorder, based upon data from the patient (e.g., from repeat datasets) and/or a group of patients (e.g., of the same demographic as the patient, of a different demographic to the patient). Furthermore, scores from the aggregate dataset can be paired prior to reduction according to any other the above methods, whereby pairing can be performed based upon identification of a positive correlation between scores from each of the interview and the observation datasets, a negative correlation between scores from each of the interview and the observation datasets, or no correlation between scores from each of the interview and the observation datasets. One or more embodiments of the first variation include weighting, weighting can be performed using a measure of variance (e.g., standard deviation, correlation, variance, etc.) between items grouped according to behavioral category, paired according to correlation, grouped according to redundancy, or grouped by any other suitable means, in order to determine an appropriate weight as a measure of confidence. Then, a determined weight can be multiplied with the score(s) during reduction to form the aggregate reduced dataset. In these variations “higher weights” can be greater than zero or one, and “lower weights” can be less than one or zero. In examples of weighting, a positive correlation can be used to attribute a higher weight or a lower weight to one or more items that are positively correlated, a negative correlation can be used to attribute a higher weight, a lower weight, or a weight of zero to one or more items that are negatively correlated, a zero correlation can be used to attribute a lower weight or a weight of zero to non-correlated items, a lower weight or a weight of zero can be attributed to grouped items that have high variability, and a higher weight or a lower weight can be attributed to grouped items that have low variability. Reduction can thus be performed based upon analysis of the aggregate dataset, the interview dataset, and/or the observation dataset, and can additionally or alternatively be performed based upon historical data pertaining to demographics including, similar to, and/or different from the patient.

In one or more embodiments, reduction of the interview dataset and the observation dataset is performed prior to generation of the aggregate reduced dataset. The reduction of the interview dataset and the observation dataset in the second variation is performed based upon analysis of historical data for demographics including, similar to, and/or different from the patient. In one or more embodiments, the reduction is based upon data reduction techniques including one or more of: alternating decision tree analysis, best-first decision tree analysis, decision stump tree analysis, functional tree analysis, C4.5 decision tree analysis, repeated incremental pruning analysis, logistic alternating decision tree analysis, logistic model tree analysis, nearest neighbor generalized exemplar analysis, association analysis, divide-and-conquer analysis, random tree analysis, decision-regression tree analysis with reduced error pruning, ripple down rule analysis, classification and regression tree analysis, and any other suitable reduction analysis technique. In the second variation, the reduction is performed using the same reduction technique(s) for each of the interview dataset and the observation dataset separately prior to aggregation of the reduced datasets; however, the reduction can be performed using different techniques for each of the interview dataset and the observation dataset prior to aggregation.

In one or more embodiments, aggregating the reduced interview and observation datasets can include grouping items of the reduced datasets based upon any one or more of: similarity in observed behavior, positive correlation in quantified score, negative correlation in quantified score, no correlation in quantified score, an identified importance, and any other suitable factors. Alternatively, aggregation can be performed without grouping, and/or can include any one or more of: adding scores, weighting scores (e.g., based upon importance, based upon a measure of variance), subtracting scores, averaging scores, omitting scores, and any other suitable mathematical operation as described in relation to the first variation above. Finally, in some variations of the second variation a secondary reduction can be performed to arrive at the aggregate reduced dataset, which can include any one or more of: omission, weighting, subtraction, adding, and averaging of scores for redundant, important, or non-important items.

In one or more embodiments, reducing at least one of the interview dataset and the observation dataset according to historical or non-historical data from the patient or demographic of patients can include implementation of a machine learning algorithms, which can be trained based upon data from the patient and/or data from demographics including, similar to, and/or different from the patient. As such, by accumulation of data and machine learning, identification of items known to correlate with each other, known to compete with each other known to negate each other, known to be indicative, alone or in combination, of a disorder state, and/or known to have some importance in any other suitable manner can contribute to generation of the aggregate reduced dataset. Furthermore, aggregation and/or reduction can be performed according to any suitable combination of the above-described methods, and can be performed any suitable number of times and in any suitable order.

In one or more embodiments, the calculation of a value of a metric derived from the aggregate reduced dataset, can function to derive at least one value of a metric for comparison to a set of criteria for determining a state of the disorder for the patient. Calculating the value of the metric can including any one or more of: averaging, adding, and weighting (e.g., based upon a measure of variance) all or a subset of the aggregate reduced dataset, with or without grouping based upon a common feature (e.g., behavior category).

In one or more embodiments, grouping and calculating the value of the metric preferably includes calculating a value for each of a set of metrics, including at least one metric for each group (e.g., behavior category) characterized in the aggregate reduced dataset. Every value of a metric of the set of metrics is preferably determined in an identical manner using one or more of the above described techniques; however, one or more values of metrics of the set of metrics can alternatively be determined in a manner different from that of another value of a metric of the set of metrics. In relation to calculating a value of a metric for the aggregate reduced dataset, the value of the metric can be a value of a representative metric derived from the set of metrics determined for the set of groups. For instance, in some variations, all values for the set of metrics corresponding to behavior categories can be added together or averaged in order to determine a value of a representative metric. In one such example for Autism Spectrum Disorder characterization, with regard to the ADI-R and the ADOS, scores of the aggregate reduced dataset corresponding to different behavior categories (e.g., social interaction, communication and language, restricted and repetitive behaviors, etc.) can be averaged to calculate a value of a metric for each behavior category. Then, the average of the values of the metrics for the behavior categories can be determined as the representative value for the patient. Alternatively, however, calculating the value of the metric(s) can be performed in any other suitable manner.

Additionally, a set of instructions may be provided to an entity capturing the video dataset, which guide an entity in documenting elements of the observation dataset to increase the efficiency of characterizing and/or diagnosing one or more disorders of the patient. For example, the entity can be any entity who is associated well enough with the patient to reliably capture and/or prompt behaviors of the patient. In variations, the entity can be any one or more of: a parent, a sibling, a healthcare provider, a supervisor, a peer, a layperson, a professional person, or any other suitable entity. The set of instructions preferably guide the entity in administering tasks or activities to the patient to prompt at least one behavior (e.g., flapping, repetitive motion, pointing, showing, social interaction behavior, emotional response behavior, attention behavior, etc.) that can be used to characterize/diagnose the disorder, but can additionally or alternatively guide the entity in passively capturing behaviors of the patient.

As shown in FIG. 4, a process flow is shown where patient profile 412 is employed to generate one or more goal models 414, which employs the assessment data to train goal models to generate one or more goals. The goals are used to generate one or more therapy models 416, which employs the goals to train the therapy models to generate one or more therapies personalized for the patient. At block 418, the one or more therapies are performed by one or more of a professional person, a layperson, a peer, a therapy device, an entity, or the like. Further at block 420, the results of the one or more therapies are provided for analysis. In one or more embodiments, one or more professional persons, laypersons, peers, entities, or third parties, may provide the results, analysis the results, modify the results, provide result annotations, or the like.

When one or more of the results of the one or more therapies do not match the one or more goals, the one or more goal models 414 are retrained with the one or more unmatched therapy results and the received assessment data to provide one or more retrained goals to therapy models 418. The one or more retrained goals and the one or more therapy results are employed to retrain one or more therapy models 416 to generate one or more retrained therapies which are performed. The one or more results of the retrained therapies are analyzed at block 420. If the one or more retrained goals match the one or more results of the retrained therapies, reports 422 are generated and provided to a user of the patient management system. However, if the one or more retrained goals match the one or more results of the retrained therapies, the process may iteratively repeat the retraining steps until one or more matches occur.

In one or more embodiments, one or more of a professional person, a layperson, a peer, a third party, an entity, or the like may decide when enough iterations of retrained therapies, if any, have been performed with the patient without having to wait for one or more matches to actually occur.

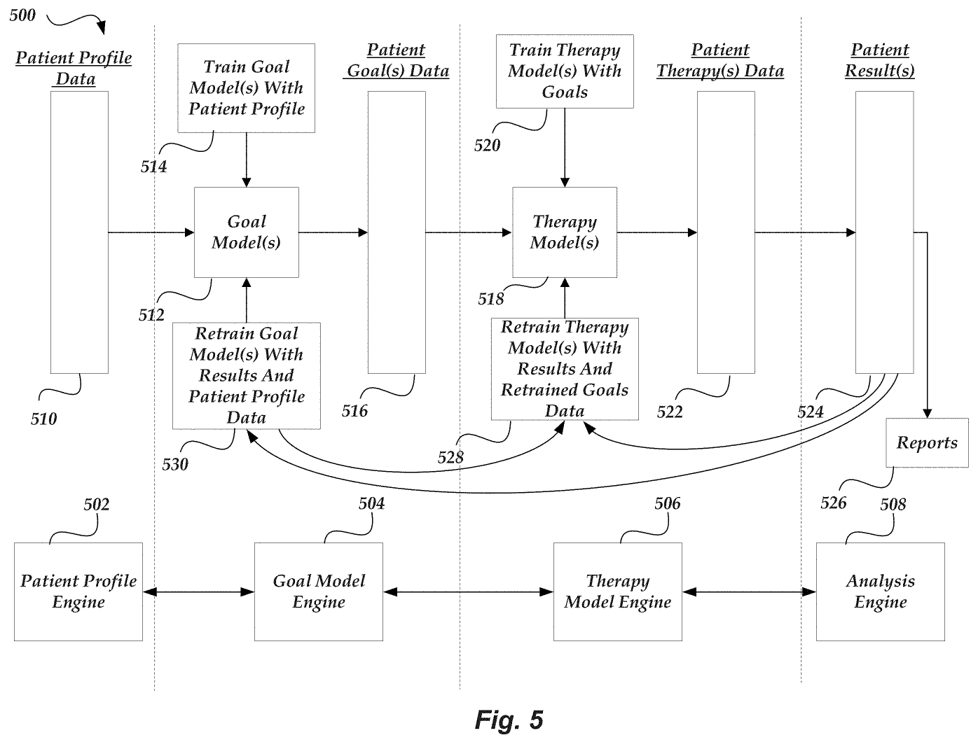

FIG. 5 illustrates a logical schematic of at least a portion of platform 500 for managing Machine Learning (ML) operations with models, engines, and applications that provide personalized goals and therapies that treat one or more disorders of a patient. In one or more of the various embodiments, system 500 may be hosted by one or more network computers, such as, as network computer 300 or client computer 200, etc.

As shown, patient profile engine 502 is employed to receive different types of assessment data and generate at least one or more diagnosis and normalized assessment data that is included in patient profile data 510 which is subsequently provided to goal model engine 504. In one or more embodiments, one or more diagnosis models may be generated and trained with at least the normalized assessment data. The training of the one or more diagnosis models generates one or more diagnosis for the patient.

Goal model engine 504 performs several different actions as shown. For example, the diagnosis and normalized assessment data is employed to generate one or more goal models 512. Also, one or more goal models 512 are trained 514 with information included in patient profile 510, which includes at least the diagnosis and normalized assessment data. The training of the goal models generates one or more personalized goals 516 that can be used to indicate one or more therapy results in the treatment of the one or more disorders of the patient.

Therapy model engine 506 performs several different actions as shown. For example, the one or more personalized goals are employed to generate one or more therapy models 518. Also, one or more therapy models 518 are trained 520 with the one or personalized goals 516. The training of the therapy models generates one or more personalized patient therapies 522 for treating the one or more disorders of the patient. In one or more embodiments, one or more of a professional person, a layperson, a peer, a therapy device, third party, or the like, may be employed to provide the one or more personalized therapies to the patient. Also, one or more of a professional person, a layperson, a peer, a therapy device, a third party, or the like, may be employed to provide the one or more therapy results for the patient. In one or more embodiments, a patient therapy result may be provided in a report to at least the provider of the therapy and as data to an analysis engine.

Analysis engine performs several actions as shown. For example, one or more patient results 524 for treating the patient with the one or more therapies 522 is compared to the one or more personalized goals 516. If the comparison indicates a match within one or more thresholds of similarity, equivalence, and/or range between the one or more goals and the one or more patient results for the one or more therapies performed with the patient, then reports 526 are provided indicating the current results and predicted results for further treatment of the patient by the one or more currently performed therapies, new therapies, or retrained therapies.

However, if the comparison indicates there is not a match within one or more thresholds of similarity or equivalence between the one or more goals and the one or more results for the one or more therapies performed with the patient, then goal model engine 504 retrains the one or more goal models to produce one or more retrained goals 530 based on the one or more therapy results and the patient profile data. In one or more embodiments, one or more of a professional person, a layperson, a peer, a third party, or the like may decide when enough iterations of retrained therapies, if any, have been performed with the patient without having one or more matches occur.

Additionally, therapy model engine 506 retrains the one or more therapy models with the one or more retrained goals and the one or more therapy results 528 to generate one or more retrained therapies that is performed with the patient. This retraining process may continue until the one or more therapy results match within one or more thresholds of similarity or equivalence between the one or more retrained goals and the one or more results for the one or more retrained therapies performed with the patient. In one or more embodiments, one or more professional persons, laypersons, peers, entities, or third parties, may provide the results, analysis the results, modify the results, provide result annotations, or the like.

Also, in one or more embodiments, a library of one or more models for diagnosis, goals, and therapies may be employed to provide at least a template for the one or more models generated for a patient. The library may include previously generated models for patients, or new models provided by third party entities. Also, models that are determined to not result in somewhat matching goals and therapy results may be modified to improve the probability of matching or removed from the library.

### Generalized Operations

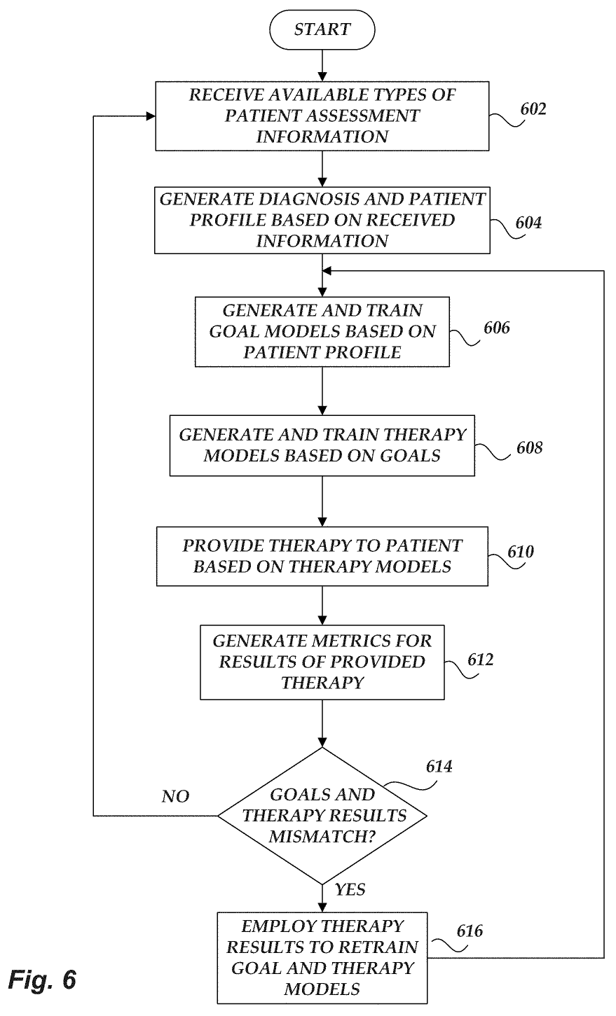

FIG. 6 shows a flowchart for an ML platform that generates and trains goal models and therapy models to provide therapy results that converge with goals. Moving from a start block, the process advances to block 602 where available assessment data regarding a patient is received. In one or more embodiments, different types of available assessment data for a patient may be received including one or more of layperson assessment data, clinical assessment data, biometric assessment data provided by a biometric device, video assessment data, medical assessment data, heuristic data and the like.

In block 604, a patient profile is generated for the patient based on the received assessment data and the heuristic data. Additionally, one or more different weights may be associated with one or more different types of the received assessment data. The available/received different types of assessment data and their associated weights are employed to generate a diagnosis of one or more neuropsychiatric and/or neurological disorders that the patient. In one or more embodiments, one or more diagnosis models may be generated and trained with at least the different types of received assessment data. The training of the one or more diagnosis models generates one or more diagnosis for the patient.

At block 606, one or more goal models are generated and trained based on the different types of received assessment data, diagnosis, heuristic data and other information included in the patient profile. The one or more trained goal models are used to generate one or more goals. Alternatively, one or more of the one or more goals may be separately provided by one or more of a layperson, a peer, a professional person, an entity, or a third party.

At block 608, the one or more goals are employed to generate and train one or more therapy models. The trained one or more therapy models employ the one or more goals to generate one or more therapies to be provided to the patient. Alternatively, one or more of the therapies may be separately provided by one or more of a layperson, a peer, a professional person, an entity, or a third party.

At block 610, the patient is treated with the one or more therapies by one or more of a layperson, a peer, a professional person, a therapy device, an entity, or a third party. Also, one or more results for the one or more personalized therapies are provided by one or more of a layperson, a professional person, a peer, a therapy device, an entity, or a third party.

At block 612, metrics for the results for treating the one or more patients with the one or more therapies is compared to the one or more personalized goals.

At decision block 614, if the comparison indicates a match within one or more thresholds of similarity, equivalence, and/or range between the one or more personalized goals and the one or more patient results for the one or more personalized therapies performed with the patient, then the process moves to block 602 where the one or more patient results are employed to update the patient profile and the process is ready to perform substantially the same actions again with the updated patient profile.

However, if the comparison, at decision block 614, does not indicate a match, then the process flows back to block 606. The the one or more goal models are retrained to produce one or more retrained goals based on the one or more non-matching therapy results and the patient profile data. Also, the one or more therapy models are retrained with the one or more retrained goals and the one or more non-matching therapy results to generate one or more retrained therapies that is performed with the patient.

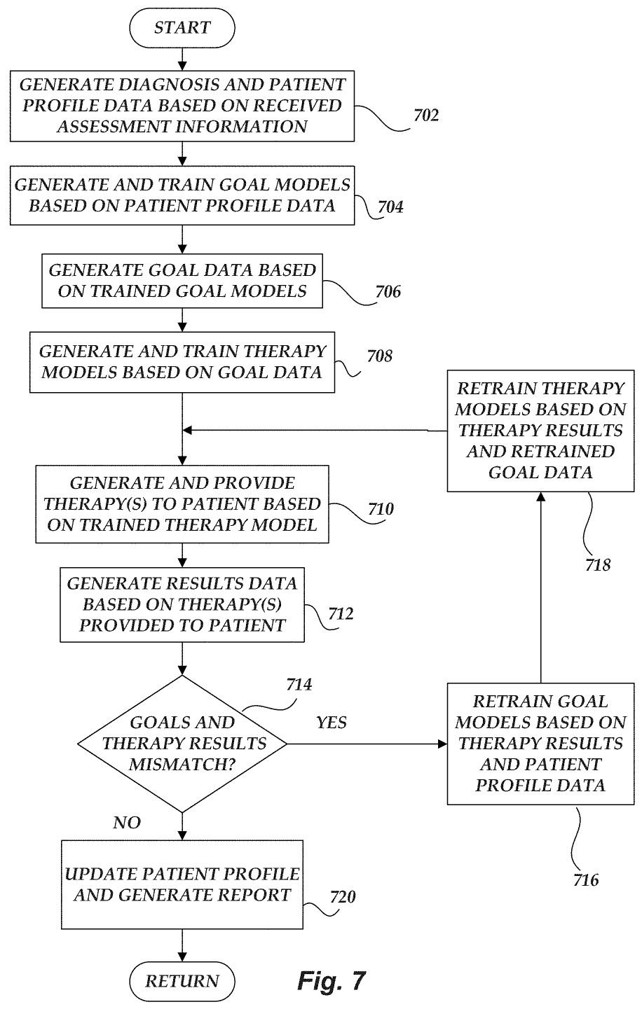

FIG. 7 illustrates a flowchart for a process for an ML platform that generates patient profiles that are employed to train and retrain models until therapy results converge with one or more goals.

Moving from a start block, the process advances to block 702 where available assessment data regarding a patient is received. In one or more embodiments, different types of available assessment data for a patient may be received including one or more of layperson assessment data, clinical assessment data, biometric assessment data provided by a biometric device, video assessment data, medical assessment data, heuristic data and the like.

In block 704, a patient profile is generated for the patient based on the received assessment data and the heuristic data. Additionally, one or more different weights may be associated with one or more different types of the received assessment data. The available/received different types of assessment data and their associated weights are employed to generate a diagnosis of one or more neuropsychiatric and/or neurological disorders that the patient. In one or more embodiments, one or more diagnosis models may be generated and trained with at least the normalized assessment data. The training of the one or more diagnosis models generates one or more diagnosis for the patient.

At block 706, one or more goal models are generated and trained based on the different types of received assessment data, diagnosis, heuristic data, and other information included in the patient profile. The one or more trained goal models generate one or more goals. Alternatively, one or more of the goals may be separately provided by one or more of a layperson, a professional person, a peer, an entity, or a third party.

At block 708, the one or more goals are employed to generate and train one or more therapy models. The trained one or more therapy models employ the one or more goals to generate one or more therapies to be provided to the patient. Alternatively, one or more of the one or more therapies may be separately provided by one or more of a layperson, a peer, a professional person, an entity, or a third party.

At block 710, the patient is treated with the one or more therapies by one or more of a layperson, a professional person, a peer, a therapy device, an entity, or a third party.

At block 712, one or more results and/or metrics for treating the one or more patients with the one or more therapies is generated. Also, one or more results for the one or more personalized therapies are provided by one or more of a layperson, a peer, a professional person, a therapy device, or a third party, which may provide and/or observe the one or more therapies provided to the patient.

At decision block 714, if the comparison indicates a match within one or more thresholds of similarity, equivalence, and/or range between the one or more personalized goals and the one or more patient results for the one or more personalized therapies performed with the patient, then the process moves to block 720 where the one or more patient results are employed to update the patient profile and the process is ready to perform substantially the same actions again with the updated patient profile. Also, a report may be provided regarding the one or more results, the one or more therapies, or the one or more goals, or the updated patient profile. The updated patient profile may include one or more new diagnosis based on the successful matching of the one or more goals and the one or more therapy results. The process returns to performing other actions.

However, if the comparison, at decision block 714, does not indicate a match, then the process flows to block 716, where the one or more goal models are retrained to produce one or more retrained goals based on the one or more non-matching therapy results and the patient profile data. Further, the process advances to block 718 where the one or more therapy models are retrained with the one or more retrained goals and the one or more non-matching therapy results to generate one or more retrained therapies that is performed with the patient. Next, the process returns to block 710 where substantially the same actions are iteratively performed again with the one or more retrained therapies.

### Graphical User Interfaces For Apps

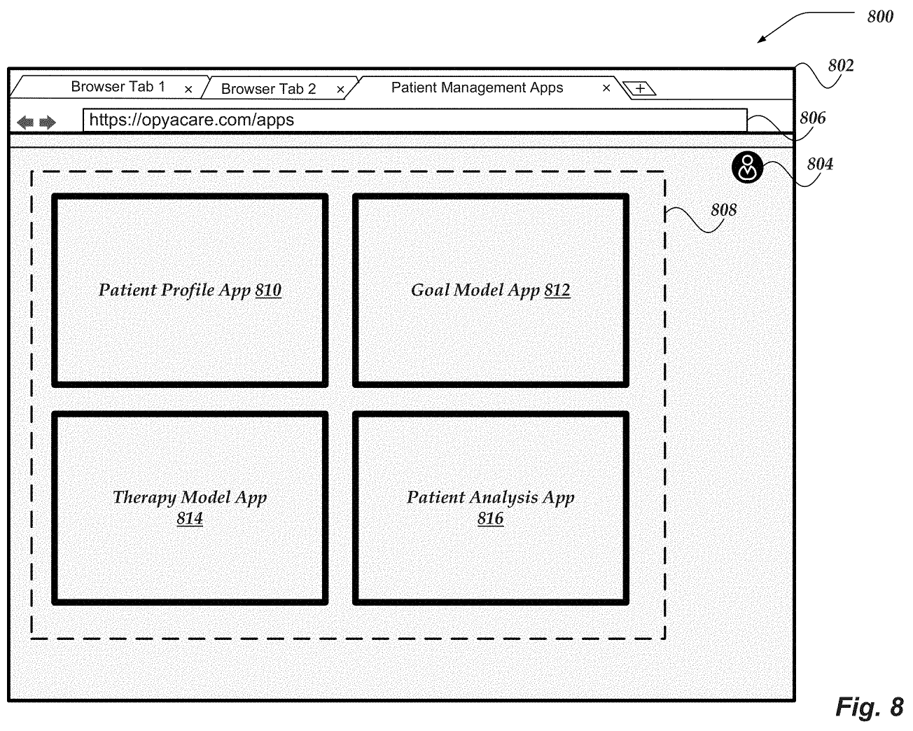

FIG. 8 shows user interface 800 for selecting patient management applications. In the figure, user interface 800 is resolved in webpage 802 which includes navigation tabs and search bar 806. Also, icon 804 identifies when an authorized user is accessing webpage 802. Display space 808 includes four applications including patient profile app 810, goal model app 812, therapy model app 814, and patient analysis app 816.

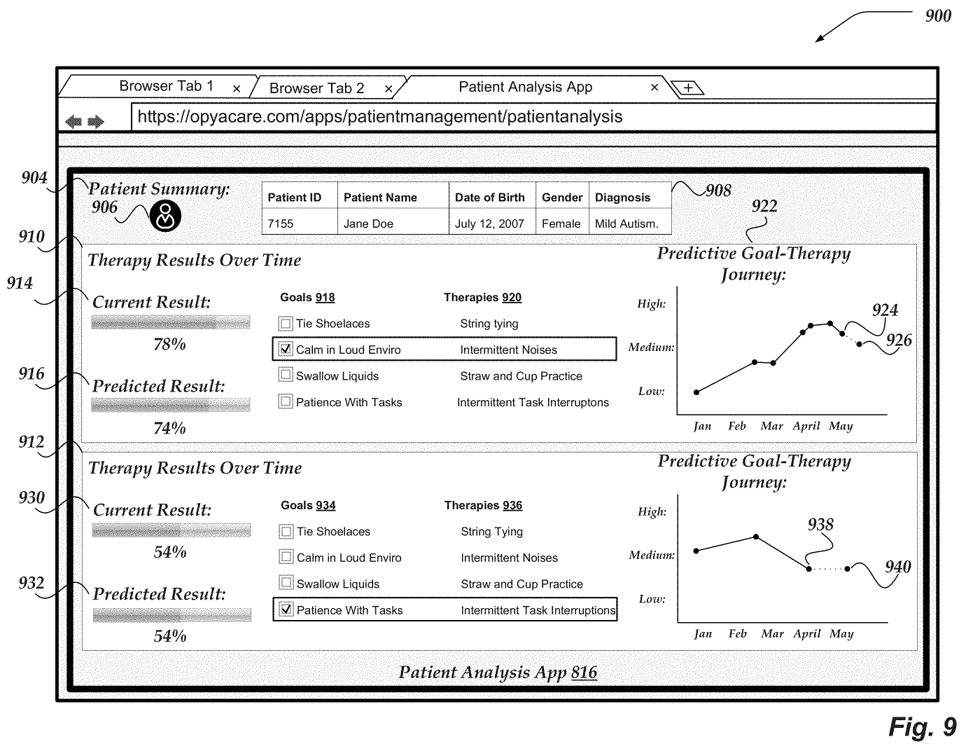

FIG. 9 shows user interface 900 for patient analysis application 816. As illustrated, application 816 includes patient summary 904, table patient summary 908, authorized person icon 906, Therapy results over time list 910 for a selected goal of calm in loud environment and a selected therapy of intermittent noises. List 910 includes current result 914, predicted result 916, goals 918, therapies 920, predictive goal therapy journey list 922, graphed current therapy data point 924, and graphed predicted therapy data point 926. Another therapy results over time list 912 includes a selected goal of patience with tasks and a selected therapy of intermittent task interruptions. List 912 also includes another current result 930, another predicted result 932, another goals 934, another therapies 936, another graphed current therapy data point 938 and another graphed predicted data point 940.

Additionally, although not shown, patient analysis application 816 can include any one or more of: an analysis of an expected progress according to the one or more goals and/or the one or more therapies, a comparison between the expected progress and the actual progress of the patient, an analysis of non-responsiveness to the treatment plan, an analysis of a detrimental response to the treatment plan by the patient, an analysis of potential substitutions, subtractions, or additions to the treatment plan (e.g., alternative therapies, alternative medications, unrecommended therapies, unrecommended medications, etc.), and any other suitable analysis of a parameter indicative of progress. The analysis can then be used to modify the patient profiled, goals and/or therapies for the patient, followed by subsequent monitoring of the progress of the patient.

It will be understood that each block of the flowchart illustration, and combinations of blocks in the flowchart illustration, can be implemented by computer program instructions. These program instructions may be provided to a processor to produce a machine, such that the instructions, which execute on the processor, create means for implementing the actions specified in the flowchart block or blocks. The computer program instructions may be executed by a processor to cause a series of operational steps to be performed by the processor to produce a computer-implemented process such that the instructions, which execute on the processor to provide steps for implementing the actions specified in the flowchart block or blocks. The computer program instructions may also cause at least some of the operational steps shown in the blocks of the flowchart to be performed in parallel. Moreover, some of the steps may also be performed across more than one processor, such as might arise in a multi-processor computer system. In addition, one or more blocks or combinations of blocks in the flowchart illustration may also be performed concurrently with other blocks or combinations of blocks, or even in a different sequence than illustrated without departing from the scope or spirit of the invention.

Accordingly, blocks of the flowchart illustration support combinations of means for performing the specified actions, combinations of steps for performing the specified actions and program instruction means for performing the specified actions. It will also be understood that each block of the flowchart illustration, and combinations of blocks in the flowchart illustration, can be implemented by special purpose hardware based systems, which perform the specified actions or steps, or combinations of special purpose hardware and computer instructions. The foregoing example should not be construed as limiting or exhaustive, but rather, an illustrative use case to show an implementation of at least one of the various embodiments of the invention.

Further, in one or more embodiments (not shown in the figures), the logic in the illustrative flowcharts may be executed using an embedded logic hardware device instead of a CPU, such as, an Application Specific Integrated Circuit (ASIC), Field Programmable Gate Array (FPGA), Programmable Array Logic (PAL), or the like, or combination thereof. The embedded logic hardware device may directly execute its embedded logic to perform actions. In one or more embodiment, a microcontroller may be arranged to directly execute its own embedded logic to perform actions and access its own internal memory and its own external Input and Output Interfaces (e.g., hardware pins or wireless transceivers) to perform actions, such as System On a Chip (SOC), or the like.

What is claimed as new and desired to be protected by Letters Patent of the United States is:

1. A method for managing treatment for a patient, wherein one or more processors execute instructions to perform actions, comprising:
instantiating a patient management engine that performs actions, including:
receiving assessment information that is employed to generate one or more diagnoses of one or more disorders for the patient;
generating a profile of the patient based on one or more of the one or more diagnoses, the received information, or one or more other profiles previously generated for one or more other patients;
generating one or more goal models based on the profile, wherein the one or more goal models are trained with the received assessment information, and wherein the trained goal models are employed to generate one or more goals for the patient;
generating one or more therapy models based on one or more of the one or more goals or one or more other therapy models previously generated for the one or more other patients, wherein the one or more therapy models are trained with the one or more goals;
employing the one or more trained therapy models to generate one or more therapies for the patient, wherein one or more results for the one or more therapies are provided;
in response to the one or more results mismatching with the one or more goals, iteratively performing further actions until a match occurs, including:
retraining the one or more goal models with the one or more mismatched therapy results and the profile, wherein the one or more retrained goal models generate retrained goals for the patient;
retraining the one or more trained therapy models with one or more retrained goals, wherein the one or more retrained therapy models generate one or more retrained therapies for the patient; and
in response to a result matching a goal, updating the profile and providing a report.

2. The method of claim 1, further comprising providing metrics for the one or more results based on one or more of participation, non-participation, or completion by the patient of the one or more therapies over time.

3. The method of claim 1, further comprising generating one or more predictive results for the one or more therapies based on one or more of the profile, another profile previously provided for another patient, a therapy model, or another therapy models.

4. The method of claim 1, wherein the training of the one or more therapy models further comprises employing one or more other goals that is provided by one or more of a family relative, a care giver, or a third party.

5. The method of claim 1, wherein generating the one or more diagnoses further comprises separately weighting different types of received assessment information.

6. The method of claim 1, wherein the received assessment information further comprises different types of assessment information, including one or more of cohort data, clinical data, biometric device data, or video data.

7. The method of claim 1, further comprising providing a library for a plurality of the other patient profiles, the other goal models, and the other therapy models, wherein one or more portions of the library are further employed in retraining the one or more goal models and the one or more therapy models.

8. The method of claim 1, further comprising providing one or more of a patient profile app, a goal model app, a therapy model app, or a patient analysis app, wherein each app provides a graphical user interface to enable editing and displaying information.

9. The method of claim 1, further comprising providing a graphical user interface that displays analysis of the one or more goals, the one or more therapies, the one or more results, the one or more diagnoses, and one or more predictive results.

10. A system for managing treatment for a patient, comprising:
a memory for storing instructions; and
one or more processors that execute the instructions to perform actions, including:
instantiating a patient management engine that performs actions, including:
receiving assessment information that is employed to generate one or more diagnoses of one or more disorders for the patient;
generating a profile of the patient based on one or more of the one or more diagnoses, the received information, or one or more other profiles previously generated for one or more other patients;
generating one or more goal models based on the profile, wherein the one or more goal models are trained with the received assessment information, and wherein the trained goal models are employed to generate one or more goals for the patient;
generating one or more therapy models based on one or more of the one or more goals or one or more other therapy models previously generated for the one or more other patients, wherein the one or more therapy models are trained with the one or more goals;
employing the one or more trained therapy models to generate one or more therapies for the patient, wherein one or more results for the one or more therapies are provided;
in response to the one or more results mismatching with the one or more goals, iteratively performing further actions until a match occurs, including:
retraining the one or more goal models with the one or more mismatched therapy results and the profile, wherein the one or more retrained goal models generate retrained goals for the patient;
retraining the one or more trained therapy models with one or more retrained goals, wherein the one or more retrained therapy models generate one or more retrained therapies for the patient; and
in response to a result matching a goal, updating the profile and providing a report.

11. The system of claim 10, further comprising providing metrics for the one or more results based on one or more of participation, non-participation, or completion by the patient of the one or more therapies over time.

12. The system of claim 10, further comprising generating one or more predictive results for the one or more therapies based on one or more of the profile, another profile previously provided for another patient, a therapy model, or another therapy models.

13. The system of claim 10, wherein the training of the one or more therapy models further comprises employing one or more other goals that is provided by one or more of a family relative, a care giver, or a third party.

14. The system of claim 10, wherein generating the one or more diagnoses further comprises separately weighting different types of received assessment information.

15. The system of claim 10, wherein the received assessment information further comprises different types of assessment information, including one or more of cohort data, clinical data, biometric device data, or video data.

16. The system of claim 10, further comprising providing a library for a plurality of the other patient profiles, the other goal models, and the other therapy models, wherein one or more portions of the library are further employed in retraining the one or more goal models and the one or more therapy models.

17. The system of claim 10, further comprising providing one or more of a patient profile app, a goal model app, a therapy model app, or a patient analysis app, wherein each app provides a graphical user interface to enable editing and displaying information.

18. The system of claim 10, further comprising providing a graphical user interface that displays analysis of the one or more goals, the one or more therapies, the one or more results, the one or more diagnoses, and one or more predictive results.

19. A processor readable non-transitory storage media that includes instructions for managing treatment of a patient, wherein execution of the instructions by one or more processors performs actions, comprising:
instantiating a patient management engine that performs actions, including:
receiving assessment information that is employed to generate one or more diagnoses of one or more disorders for the patient;
generating a profile of the patient based on one or more of the one or more diagnoses, the received information, or one or more other profiles previously generated for one or more other patients;
generating one or more goal models based on the profile, wherein the one or more goal models are trained with the received assessment information, and wherein the trained goal models are employed to generate one or more goals for the patient;
generating one or more therapy models based on one or more of the one or more goals or one or more other therapy models previously generated for the one or more other patients, wherein the one or more therapy models are trained with the one or more goals;
employing the one or more trained therapy models to generate one or more therapies for the patient, wherein one or more results for the one or more therapies are provided;
in response to the one or more results mismatching with the one or more goals, iteratively performing further actions until a match occurs, including:
retraining the one or more goal models with the one or more mismatched therapy results and the profile, wherein the one or more retrained goal models generate retrained goals for the patient;
retraining the one or more trained therapy models with one or more retrained goals, wherein the one or more retrained therapy models generate one or more retrained therapies for the patient; and
in response to a result matching a goal, updating the profile and providing a report.

20. The processor readable non-transitory storage media of claim 19, further comprising providing metrics for the one or more results based on one or more of participation, non-participation, or completion by the patient of the one or more therapies over time.

21. The processor readable non-transitory storage media of claim 19, further comprising generating one or more predictive results for the one or more therapies based on one or more of the profile, another profile previously provided for another patient, a therapy model, or another therapy models.

22. The processor readable non-transitory storage media of claim 19, wherein the training of the one or more therapy models further comprises employing one or more other goals that is provided by one or more of a family relative, a care giver, or a third party.

23. The processor readable non-transitory storage media of claim 19, wherein generating the one or more diagnoses further comprises separately weighting different types of received assessment information.

24. The processor readable non-transitory storage media of claim 19, wherein the received assessment information further comprises different types of assessment information, including one or more of cohort data, clinical data, biometric device data, or video data.

25. The processor readable non-transitory storage media of claim 19, further comprising providing a library for a plurality of the other patient profiles, the other goal models, and the other therapy models, wherein one or more portions of the library are further employed in retraining the one or more goal models and the one or more therapy models.

26. The processor readable non-transitory storage media of claim 19, further comprising providing one or more of a patient profile app, a goal model app, a therapy model app, or a patient analysis app, wherein each app provides a graphical user interface to enable editing and displaying information.

27. The processor readable non-transitory storage media of claim 19, further comprising providing a graphical user interface that displays analysis of the one or more goals, the one or more therapies, the one or more results, the one or more diagnoses, and one or more predictive results.
